(19) United States
(12) Patent Application Publication
(10) Pub. No.: US 2026/0337323 A1
(43) Pub. Date: Oct. 29, 2026

(54) COMPOSITIONS INCLUDING A MIXTURE OF ISOMERS OF ITACONIMIDE NORBORNENE AND CITRACONIMIDE NORBORNENE

(71) Applicant: 3M INNOVATIVE PROPERTIES COMPANY, St. Paul, MN (US)

(72) Inventors: Michael W. Kobe, Lake Elmo, MN (US); Gregory P. Sorenson, Maplewood, MN (US); Erik M. Townsend, South St. Paul, MN (US); Claire Hartmann-Thompson, Lake Elmo, MN (US); Ralph R. Roberts, Oakdale, MN (US); Mark J. Pellerite, Woodbury, MN (US); Phillip D. Hustad, St. Paul, MN (US)

(21) Appl. No.: 19/108,474

(22) PCT Filed: Sep. 26, 2023
(86) PCT No.: PCT/IB2023/059546

§ 371 Date: Mar. 4, 2025

Related U.S. Application Data
(60) Provisional application No. 63/420,899, filed on Oct. 31, 2022.

(51) Int. Cl. C08F 222/40; C07D 207/448; C08K 11/00

(52) U.S. Cl. CPC C08F 222/40; C07D 207/448; C08K 11/00

(57) ABSTRACT

Compositions are provided, including a mixture of isomers of itaconimide norbornene and citraconimide norbornene. Copolymers can be formed using the compositions and optionally at least one additional norbornene monomer. Additionally, films can be prepared that include a crosslinked reaction product of any of the copolymers. The compositions, copolymers, and films may have useful dielectric properties.

### BACKGROUND

The current state of the art in low dielectric materials is represented by fluoropolymers such as poly(tetrafluoroethylene) (PTFE) and poly(hexafluoropropylene-tetrafluoroethylene) (fluorinated ethylene/propylene or FEP). These materials offer in some instances a dielectric constant (Dk) of <2.0 and a dissipation factor (Df) of <0.001, but at high cost and with processing difficulties such as extremely high processing temperatures (above 250° C. for FEP and above 350° C. for PTFE) and high melt viscosities. Hydrocarbon polymers can offer low Dk/Df performance, but materials such as polyethylene, polypropylene, and styrenics suffer from low use temperatures.

### SUMMARY

In a first aspect, a composition is provided. The composition comprises a mixture of isomers comprising:
at least 10% by weight of isomer A, which has the following formula:

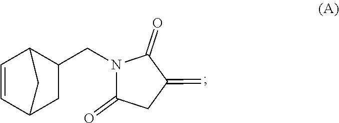 [SMILES: C=C1CC(=O)N(CC2CC3C=CC2C3)C1=O]

and
up to 90% by weight of isomer B, which has the following formula:

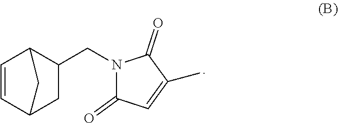 [SMILES: CC1=CC(=O)N(CC2CC3C=CC2C3)C1=O]

In a second aspect, a copolymer is provided. The copolymer is a reaction product of a polymerizable composition comprising:

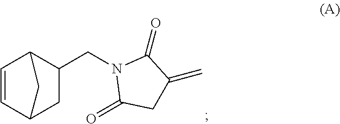 [SMILES: C=C1CC(=O)N(CC2CC3C=CC2C3)C1=O]

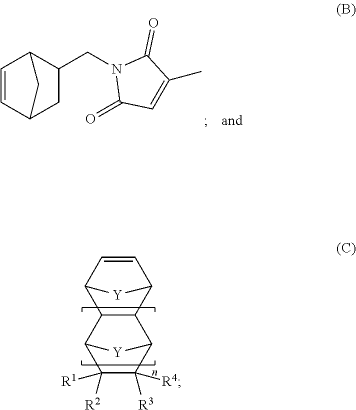

wherein each of R¹, R², R³, and R⁴ is independently selected from H, a linear or branched C1 to C20 hydrocarbyl; a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

In a third aspect, another copolymer is provided. The copolymer comprises the following divalent monomer units:

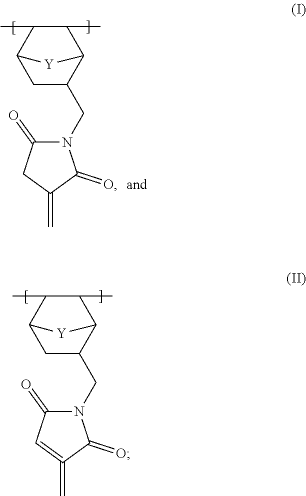

wherein each Y is independently selected from —CH₂—, —CH₂CH₂—, and O.

In a fourth aspect, a further copolymer is provided. The copolymer comprises the following divalent monomer units:

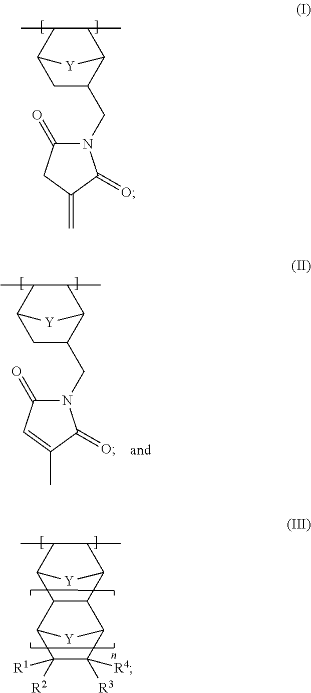

wherein each of R¹, R², R³, and R⁴ is independently selected from H, a linear or branched C1 to C20 hydrocarbyl, a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

In a fifth aspect, a composition is provided. The composition comprises the copolymer of any of the second through fourth aspects and a solvent.

In a sixth aspect, a film is provided. The film comprises a crosslinked reaction product of the copolymer of any of the second through fourth aspects.

Polymerizable compositions containing isomer A and isomer B are appropriate for use in applications requiring polymers that exhibit low dielectric constant (Dk) and dissipation factor (Df). For instance, structures incorporating copolymers prepared using isomer A and isomer B offer a means to improve thermal performance without using aromatic groups that tend to be detrimental to dielectric properties of the resulting polymer.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### DETAILED DESCRIPTION

### Glossary

The term “aliphatic” refers to C1-C40, suitably C1-C30, straight or branched chain alkenyl, alkyl, or alkynyl which may or may not be interrupted or substituted by one or more heteroatoms such as O, N, or S. The term “cycloaliphatic” refers to cyclized aliphatic C3-C30, suitably C3-C20, groups and includes those interrupted by one or more heteroatoms such as 0, N, or S. Additionally, the term “heterocyclic ring” refers to a cyclized aliphatic C5-C20, suitably C5-C10 or C5 containing one or more heteroatoms such as O, N, or S.

The term “alkyl” refers to a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 7 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkoxy” refers to a monovalent group of formula —ORa where R^{a} is an alkyl as defined above.

Each of “alkenyl” and “ene” refers to a monovalent linear or branched unsaturated aliphatic group with one or more carbon-carbon double bonds, e.g., vinyl.

The term “catenated atom” means an in-chain atom (rather than an atom of a chain substituent). The term “catenated heteroatom” means a heteroatom replaces one or more carbon atoms in a carbon chain. The heteroatom is typically oxygen, sulfur, or nitrogen.

The term “hydrocarbyl” refers to a monovalent radical of a hydrocarbon. The hydrocarbyl can be saturated, partially unsaturated, or unsaturated and can have up to 20 carbon atoms, up to 10 carbon atoms, up to 6 carbon atoms, or up to 4 carbon atoms. It often has at least 1 carbon atom or at least 2 carbon atoms. The hydrocarbyl is often an alkyl, aryl, aralkyl, or alkaryl. The term “heterohydrocarbyl” refers to a hydrocarbyl with at least one but not all of the catenated carbon atoms replaced with a heteroatom selected from O, N, or S.

The term “hydrocarbylidene” is a divalent analogue to “hydrocarbyl” in which two hydrogen atoms have been removed from the same carbon atom (e.g., such that the group bonds by a double bond). The hydrocarbylidene can be partially unsaturated or unsaturated and can have up to 20 carbon atoms, up to 10 carbon atoms, up to 7 atoms, up to 6 carbon atoms, or up to 4 carbon atoms. It often has at least 1 carbon atom or at least 2 carbon atoms. The hydrocarbylidene is often an alkylidene (═CHR).

The term “carbosilane” refers to a compound composed exclusively of Si, C, and H, and having no Si—Si bonds, suitably C1-C20, C1-C10, or C1-6.

As used herein, the term “cyclic monomer” refers to monomers having at least one cyclic group and may include bicyclics and tricyclics.

The term “aromatic” refers to C3-C40, suitably C3-C30, aromatic groups including both carbocyclic aromatic groups as well as heterocyclic aromatic groups containing one or more of the heteroatoms, O, N, or S, and fused ring systems containing one or more of these aromatic groups fused together. The term “aryl” refers to a monovalent group that is aromatic and, optionally, carbocyclic. The aryl has at least one aromatic ring. Any additional rings can be unsaturated, partially saturated, saturated, or aromatic. Optionally, the aromatic ring can have one or more additional carbocyclic rings that are fused to the aromatic ring. Unless otherwise indicated, the aryl groups typically contain from 6 to 30 carbon atoms. In some embodiments, the aryl groups contain 6 to 20, 6 to 18, 6 to 16, 6 to 12, or 6 to 10 carbon atoms. Examples of an aryl group include phenyl, naphthyl, biphenyl, phenanthryl, and anthracyl.

The term “aralkyl” refers to an alkyl group substituted with at least one aryl group. That is, the aralkyl group is of formula —R^{d}-Ar where R^{d} is an alkylene and Ar is an aryl. The aralkyl group contains 6 to 40 carbon atoms. The aralkyl group often contains an alkylene group having 1 to 20 carbon atoms or 1 to 10 carbon atoms and an aryl group having 5 to 20 carbon atoms or 6 to 10 carbon atoms.

As used herein, “C1”, “1C”, and “1 carbon” are interchangeable ways of describing a single carbon atom and may be used interchangeably when indicating any number of carbon atoms.

As used herein, the term “actinic radiation” means electromagnetic radiation of wavelength(s) capable of being absorbed by a composition exposed to it and thereby cause at least one chemical reaction or transformation to occur.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

In a first aspect, a composition is provided. The composition comprises a mixture of isomers comprising:
at least 10% by weight of isomer A, which has the following formula:

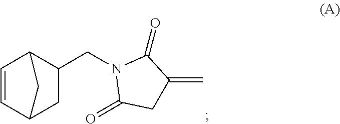 [SMILES: C=C1CC(=O)N(CC2CC3C=CC2C3)C1=O]

and

up to 90% by weight of isomer B, which has the following formula:

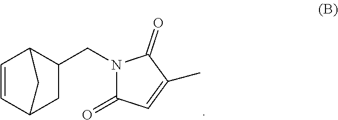 [SMILES: CC1=CC(=O)N(CC2CC3C=CC2C3)C1=O]

In a second aspect, a copolymer is provided. The copolymer is a reaction product of a polymerizable composition comprising:

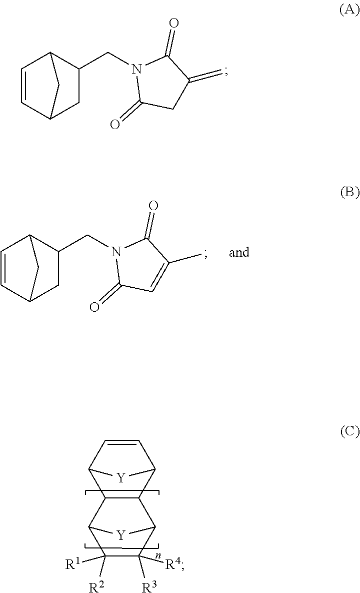

wherein each of R¹, R², R³, and R⁴ is independently selected from H, a linear or branched C1 to C20 hydrocarbyl; a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

The below disclosure relates to both the first and second aspects.

It has been discovered that it is possible to synthesize mixtures of itaconimide norbornene (i.e., isomer A) and citraconimide norbornene (i.e., isomer B) by reacting norbornene methylamine with either of itaconic anhydride or citraconic anhydride. Exemplary specific reaction and purification conditions are described in the Examples below.

The resulting isomer mixture contains at least 10% by weight (wt. %) of isomer A, such as 12 wt. % or greater, 15 wt. %, 17 wt. %, 20 wt. %, 22 wt. %, 25 wt. %, 27 wt. %, 30 wt. %, 32 wt. %, 35 wt. %, 37 wt. %, or 40 wt. % or greater; and 75 wt. % or less, 72 wt. %, 70 wt. %, 67 wt. %, 65 wt. %, 62 wt. %, 60 wt. %, 57 wt. %, 55 wt. %, 52 wt. %, 50 wt. %, 47 wt. %, 45 wt. %, 42 wt. %, 40 wt. %, 37 wt. %, 35 wt. %, 30 wt. %, 27 wt. %, or 25 wt. % or less.

The isomer mixture contains up to 90 wt. % of isomer B, such as 90 wt. % or less, 87 wt. %, 85 wt. %, 82 wt. %, 80 wt. %, 77 wt. %, 75 wt. %, 72 wt. %, 70 wt. %, 67 wt. %, 65 wt. %, 62 wt. %, or 60 wt. % or less; and 25 wt. % or greater, 27 wt. %, 30 wt. %, 32 wt. %, 35 wt. %, 37 wt. %, 40 wt. %, 42 wt. %, 45 wt. %, 47 wt. %, 50 wt. %, 52 wt. %, 55 wt. %, 57 wt. %, or 60 wt. % or greater.

In some cases, the isomer A is present in an amount of 10 wt. % to 75 wt. % of the mixture of isomers and the isomer B is present in an amount of 25 wt. % to 90 wt. % of the mixture of isomers. Often, the total of the isomer A and the isomer B is equal to 100 wt. % of the mixture of isomers. In some cases, the isomer A and the isomer B together comprise 95 wt. % to 100 wt. % of the total composition. In such embodiments, the composition may contain up to about 5 wt. % of other components, for instance additives, impurities, etc.

It is to be understood that chemical structures of substituted norbornene compounds herein are intended to encompass all exo/endo isomers and all enantiomers/diastereomers that would be consistent with the represented atom connectivity.

Compositions according to at least certain embodiments of the present disclosure may be used to prepare copolymers that are suitable for low-dielectric applications. It has been discovered that curable polynorbornene resins comprising higher amounts of pendent itaconimide functionality can be used to form copolymers that can offer enhanced thermal curing and crosslinking kinetics over systems comprising lower amounts of pendent itaconimide functionality, for instance as indicated by the storage modulus data in the Examples below.

Copolymers described herein are preparable by (e.g., prepared by) addition polymerization. As used herein, the term “addition polymerization” (also sometimes referred to in the art as vinyl-addition polymerization) refers to a polymerization process involving an olefin coordination-insertion pathway mediated by an organometallic catalyst. A schematic depiction is shown in Scheme I, below.

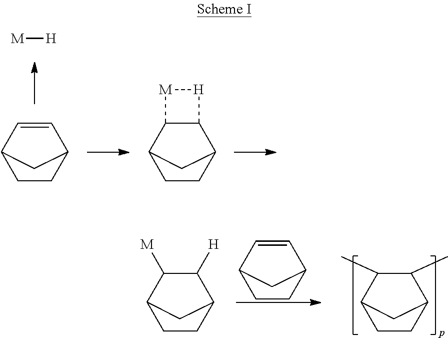

where M—H indicates an addition polymerization catalytic species having a metal hydride bond, and p represents an integer greater than 10. This process is distinguished from a common alternative polymerization method, Ring-opening Metathesis Polymerization (ROMP), both in mechanism and end product. ROMP polymers contain double bonds in the polymer backbone, whereas addition polymers according to the present disclosure do not.

Exemplary cyclic monomers suitable for addition polymerization with a mixture of isomer A and isomer B include 7-oxabicyclo[2.2.1]hept-2-ene, alkyl norbornene, cis-cyclooctene, cyclopentadiene, cyclopentene, dicyclopentadiene, hexylnorbomylene, norbomadiene, norbomylene (2-norbornene), tetracyclo[6.2.13.6.0]dodeca-4,9-diene, tetracyclopentadiene, tricyclopentadiene, and derivatives thereof with substituents including aliphatic groups, aromatic groups, esters, amides, ethers, and silanes.

Examples of suitable addition polymerizable cycloolefins comprising a ring containing a single carbon-carbon double bond include norbornene, 1-methylnorbornene, 5-methylnorbornene, 7-methylnorbornene, 5-(2-ethylhexyl)norbornene, 1-pentadecylnorbornene, 5,5-dimethylnorbornene, 5,5-dibutylnorbornene, 5,7-dibutylnorbornene, 5-methyl-5-ethylnorbornene, 5,6-didodecylnorbornene, 5-ethyl-6-propylnorbornene, 5,5,6,6-tetramethylnorbornene, 1-phenylnorbornene, 5-naphthylnorbornene, 5,5-diphenylnorbornene, 5-vinylnorbornene, 7-vinylnorbornene, 5-propenyl-6-methylnorbornene, 5-tolylnorbornene, 5-benzyl-norbornene, 5-cyclopentyl-norbornene, 1,5,5-trimethylnorbornene, 5-isopropenylnorbornene, 1-isopropyl-norbornene, 1-ethylnorbornene, 1,5-dimethyl-norbornene, 1,5-diethylnorbornene, 1,6-dimethyl-norbornene, 5,5,6-trimethylnorbornene, 5-cyclopropylnorbornene, 5-cyclohexyl-norbornene, 5-cyclopentenyl-norbornene, 5-(2′-norbomyl)norbornene, 5-phenylnorbornene, 5-benzylnorbom-2-ene, 5-(2′-phenylethyl), norbornene, 5-(3′-phenylpropyl)norbornene, 5-(4′-phenylbutyl)norbornene, 2,5-norbomadiene, cyclohexene, cyclopentene, dicyclopentadiene, 2,5-norbornadiene, bicyclo[2.2.2]-2-octene, indene, 5-methylenenorbornene, 5-ethylidenenorbornene, 5-propylidene-norbornene, 5-hexylidene-norbornene, 5-decylidenenorbornene, 5-methylene-6-methylnorbornene, 5-methylene-6-hexylnorbornene, 5-cyclohexylidenenorbornene, 5-cyclooctylidenenorbornene, 7-isopropylidenenorbornene, 5-methyl-7-isopropylidenenorbornene, 5-hydroxymethyl-6-methylenenorbornene, 7-ethylidenenorbornene, and 5-methyl-7-propylidenenorbornene.

Compositions according to the present disclosure contain one or more addition polymerization catalysts. A great many addition polymerization catalysts are known in the art and are typically based on organometallic complexes comprising Ti, Zr, Cr, Co, Fe, Cu, Ni, or Pd. Of these, addition polymerization catalysts comprising Ni or Pd are most commonly used. There is voluminous literature on organometallic addition polymerization catalysts, and especially for norbornene-type monomers. Generally, the active catalyst species is a cationic transition metal complex that has an alkyl or allyl ligand and a weakly coordinating anion. The addition polymerization catalyst may be included in polymerizable compositions according to the present disclosure as a single active species (or a combination thereof) or it may be provided as a precursor combination of a pro-catalyst and an activator; for example, as is common in the art. Generally, the pro-catalyst provides the active site for the olefin insertion mechanism that forms the addition polymer. Combination with the activator converts the pro-catalyst into its active form.

In some embodiments, appropriate catalyst(s) and pro-catalyst/activator combinations for the addition polymerization of cycloolefins comprising a ring having a single carbon-carbon bond may include Group 10 (i.e., of the Periodic Table of the Elements) catalyst(s) or pro-catalyst/activator combinations; for example, Ni-based, Pd-based, or Pt-based addition polymerization catalysts. In some preferred embodiments, late metal (e.g., Ni- or Pd-based) pro-catalysts have allyl/alkyl ligands as well as chloride ligands. These pro-catalysts are activated by the addition of monovalent metal (Li, Na, Ag) salts of weakly coordinating anions (e.g., BF₄, tetrakis(3,5-bis(trifluoromethyl)phenyl)borate (BARF), or perfluorotetraphenylborate).

Exemplary suitable pro-catalysts include: (1,1-dimethylallyl)palladium(triisopropylphosphine) trifluoroacetate, (2-chloroallyl)palladium(triisopropylphosphine) trifluoroacetate, (allyl)palladium-(tricyclohexylphosphine) chloride, (allyl)palladium(tricyclohexylphosphine) p-tolylsulfonate, (allyl)palladium(tricyclohexylphosphine) triflate, (allyl)palladium(tricyclohexylphosphine) triflimide, (allyl)palladium(tricyclohexylphosphine) trifluoroacetate, (allyl)palladium(triisopropylphosphine) triflate, (allyl)palladium(triisopropylphosphine) triflimide, (allyl)palladium(triisopropylphosphine) trifluoroacetate, (allyl)palladium(trinaphthylphosphine) triflate, (allyl)palladium(tri-o-tolylphosphine) acetate, (allyl)palladium(tri-o-tolylphosphine) nitrate, (allyl)palladium(tri-o-tolylphosphine) triflate, (allyl)palladium(triphenylphosphine) triflate, (allyl)palladium(triphenylphosphine) triflimide, (allyl)palladium(tricyclopentylphosphine) triflate, (allyl)palladium(tri-o-tolylphosphine) chloride, (allyl)Pd(AsPh₃)Cl, (allyl)Pd(PPh₃)Cl, (allyl)Pd(PCy₃)C₆F₅, (allyl)Pd(P-i-Pr₃)C₆F₅, (allyl)Pd(PMe₃)OC(O)CH₂CH═CH₂, (allyl)Pd(SbPh₃)Cl, (C₂H₅)Pd(PMe₃)₂Br, (C₂H₅)Pd(PMe₃)₂Br, (C₂H₅)Pd(PMe₃)₂Cl(Ph), (CH₃)Pd(P(i-Pr)₃)₂O₃SCF₃, (CH₃)Pd(PMe₂Ph)₂Cl, (CH₃)Pd(PMe₃)₂Cl, (CH₃)Pd(PMe₃)NO₃, (crotyl)palladium(tricyclohexylphosphine) triflate, (crotyl)palladium(tricyclopentylphosphine) triflate, (crotyl)palladium(triisopropylphosphine) triflate, (cyclooctadiene)palladium(II) dichloride, (hydrido)palladiumbis(tricyclohexylphosphine) chloride, (hydrido)palladiumbis(tricyclohexylphosphine) formate (hydrido)palladiumbis(tricyclohexylphosphine) nitrate, (hydrido)palladiumbis(tricyclohexylphosphine) triflate, (hydrido)palladiumbis(tricyclohexylphosphine) trifluoroacetate, (hydrido)palladiumbis(triisopropylphosphine) chloride, (hydrido)palladiumbis(triisopropylphosphine) triflate, (Me₂NCH₂C₆H₄)Pd(O₃SCF₃)P(cyclohexyl)₃, (methallyl)palladium(tricyclohexylphosphine) acetate, (methallyl)palladium(tricyclohexylphosphine) chloride, (methallyl)palladium(tricyclohexylphosphine) triflate, (methallyl)palladium(tricyclohexyl-phosphine) triflimide, (methallyl)palladium(tricyclohexylphosphine) trifluoroacetate, (methallyl)-palladium(tricyclopentylphosphine) acetate, (methallyl)palladium(tricyclopentylphosphine) chloride, (methallyl)palladium(tricyclopentylphosphine) triflate, (methallyl)palladium(tricyclopentylphosphine) triflimide, (methallyl)palladium(tricyclopentylphosphine) trifluoroacetate, (methallyl)palladium-(triisopropylphosphine) acetate, (methallyl)palladium(triisopropylphosphine) chloride, (methallyl)-palladium(triisopropylphosphine) triflate, (methallyl)palladium(triisopropylphosphine) triflimide, (methallyl)palladium(triisopropylphosphine) trifluoroacetate, (methallyl)Pd(AsPh₃)Cl, (methallyl)Pd(P[(OCH₂)₃]CH)Cl, (methallyl)Pd(PBu₃)Cl, (methallyl)Pd(PPh₃)Cl, (methallyl)Pd(SbPh₃)Cl, (Ph)Pd(PMe₃)₂Br, (PMe₃)₂Br, (η¹-benzyl)Pd(PEt₃)₂Cl, [(allyl)Pd(HOCH₃)(P-i-Pr₃)][B(O₂-3,4,5,6-Br₄C₆)₂], [(allyl)Pd(HOCH₃)(P-i-Pr₃)][B(O₂-3,4,5,6-Cl₄C₆)₂], [(allyl)Pd(HOCH₃)(P-i-Pr₃)][B(O₂C₆H₄)z], [(allyl)Pd(OEt₂)(PCy₃)][BF₄], [(allyl)Pd(OEt₂)(PCy₃)][PF₆], [(allyl)Pd(OEt₂)(P-iPr₃)], [(allyl)Pd(OEt₂)(P-i-Pr₃)][BPh₄], [(allyl)Pd(OEt₂)(P-i-Pr₃)][ClO₄], [(allyl)Pd(OEt₂)(PPh₃)][SbF₆], [(allyl)Pd(OEt₂)(P-i-Pr₃)][PF₆], [(allyl)Pd(OEt₂)(PPh₃)][BF₄], [(allyl)Pd(OEt₂)(PPh₃)][PF₆], [(dimethylamino)methyl]phenyl-C,N-}-palladium(tricyclohexylphosphine) triflate, [(allyl)Pd(OEt₂)(P-i-Pr₃)][BF₄], {2-[(dimethylamino)methyl]phenyl-C,N-}-palladium(tricyclohexylphosphine) chloride, dibromobis(benzonitrile)palladium(II), dichlorobis(acetonato)palladium(II), dichlorobis-(acetonitrile)palladium(II), dichlorobis(benzonitrile)palladium(II), palladium(II) bis(tricyclohexylphosphine) bis(trifluoroacetate), palladium(II) bis(tricyclohexylphosphine) diacetate, palladium(II) bis(tricyclohexylphosphine) dibromide, palladium(II) bis(tricyclohexylphosphine) dichloride, palladium(II) bis(triisopropylphosphine) bis(trifluoroacetate), palladium(II) bis(triisopropylphosphine) diacetate, palladium(II) bis(triisopropylphosphine) dibromide, palladium(II) bis(triisopropylphosphine) dichloride, palladium(II) bis(triphenylphosphine) bis(trifluoroacetate), palladium(II) bis(triphenylphosphine) diacetate, palladium(II) bis(triphenylphosphine) dibromide, palladium(II) bis(triphenylphosphine) dichloride, palladium(II) bis(tri-p-tolylphosphine) bis(trifluoroacetate), palladium(II) bis(tri-p-tolylphosphine) diacetate, palladium(II) bis(tri-p-tolylphosphine) dibromide, palladium(II) bis(tri-p-tolylphosphine) dichloride, palladium(II) ethyl hexanoate, palladium(II) acetylacetonate, palladium(II) bis(trifluoroacetate), palladium(II) ethylhexanoate, Pd(acetate)₂(PPh₃)₂, Pd(PMe₃)₂Cl, (CH₃)Pd, PdBr₂(P(p-tolyl)₃)₂, PdBr₂(PPh₃)₂, PdCl₂(P(cyclohexyl)₃)₂, PdCl₂(P(o-tolyl)₃)₂, PdCl₂(PPh₃)₂; platinum(II) chloride; platinum(II) bromide, platinum bis(triphenylphosphine)dichloride, trans-PdCl₂(PPh₃)₂, (methallyl)nickel(tricyclohexylphosphine) triflate, nickel acetylacetonate, nickel carboxylates, nickel(II) chloride, nickel(II) bromide, nickel ethylhexanoate, nickel(II) trifluoroacetate, nickel(II) hexafluoroacetylacetonate, NiCl₂(PPh₃)₂, NiBr₂P(p-tolyl)₃)₂, (allyl)platinum(tricyclohexylphosphine) chloride, (allyl)platinum(tricyclohexylphosphine) triflate, allylchloro[1,3-bis(2,6-di-i-propylphenyl)-4,5-dihydroimidazol-2-ylidene]palladium(II), allylchloro[1,3-bis(2,6-di-i-propylphenyl)imidazol-2-ylidene]palladium(II), chloro[(1,2,3-1)-3-phenyl-2-propenyl][1,3-bis(2,6-di-i-propylphenyl)-4,5-dihydroimidazol-2-ylidene]palladium(II), chloro[(1,2,3-i)-3-phenyl-2-propenyl][1,3-bis(2,6-di-i-propylphenyl)imidazol-2-ylidene]palladium(II), allylchloro[1,3-bis(2,6-di-i-propylphenyl)-4,5-dihydroimidazol-2-ylidene]nickel (II), allylchloro[1,3-bis(2,6-di-i-propylphenyl)imidazol-2-ylidene]nickel(II), chloro[(1,2,3-i)-3-phenyl-2-propenyl][1,3-bis(2,6-di-i-propylphenyl)-4,5-dihydroimidazol-2-ylidene]nickel(II), and chloro[(1,2,3-i)-3-phenyl-2-propenyl][1,3-bis(2,6-di-i-propylphenyl)imidazol-2-ylidene]nickel(II).

Addition of a Lewis base, which coordinately bonds to the metal atom, may improve the activity of addition polymerization catalysts and/or pro-catalysts. That is, the Lewis base is bonded to the metal atom by sharing both of its lone pair of electrons. Any Lewis base known in the art can be used for this purpose. Preferably, the Lewis base can dissociate readily under the polymerization conditions.

Exemplary suitable Lewis bases include substituted and unsubstituted nitriles, including alkyl nitrile, aryl nitrile or aralkyl nitrile; phosphine oxides, including substituted and unsubstituted trialkylphosphine oxides, triarylphosphine oxides, triaralkylphosphine oxides, and various combinations of alkyl, aryl and aralkylphosphine oxides; substituted and unsubstituted pyrazines; substituted and unsubstituted pyridines; phosphites, including substituted and unsubstituted trialkyl phosphites, triaryl phosphites, triaralkyl phosphites, and various combinations of alkyl, aryl and aralkyl phosphites; phosphines, including substituted and unsubstituted trialkylphosphines, triarylphosphines, triaralkylphosphines, and various combinations of alkyl, aryl, and aralkyl phosphines. Various other Lewis bases that may be used include various ethers, alcohols, ketones, amines and anilines, arsines, and stibines. In some embodiments, the Lewis base can be selected from acetonitrile, propionitrile, n-butyronitrile, tert-butyronitrile, benzonitrile (C₆H₅CN), 2,4,6-trimethylbenzonitrile, phenyl acetonitrile (C₆H₅CH₂CN), pyridine, 2-methylpyridine, 3-methylpyridine, 4-methylpyridine, 2,3-dimethylpyridine, 2,4-dimethylpyridine, 2,5-dimethylpyridine, 2,6-dimethylpyridine, 3,4-dimethylpyridine, 3,5-dimethylpyridine, 2,6-di-t-butylpyridine, 2,4-di-t-butylpyridine, 2-methoxypyridine, 3-methoxypyridine, 4-methoxypyridine, pyrazine, 2,3,5,6-tetramethylpyrazine, diethyl ether, di-n-butyl ether, dibenzyl ether, tetrahydrofuran, tetrahydropyran, benzophenone, triphenylphosphine oxide, triphenyl phosphate and PR¹₃, wherein each R¹ is independently selected from methyl, ethyl, (C₃-C₆) alkyl, substituted or unsubstituted (C₃-C₇) cycloalkyl, (C₆-C₁₀) aryl, (C₆-C₁₀) aralkyl, methoxy, ethoxy, (C₃-C₆) alkoxy, substituted or unsubstituted (C₃-C₇) cycloalkoxy, (C₆-C₁₀) aryloxy and (C₆-C₁₀) aralkyloxy.

Representative examples of PR¹₃ include trimethylphosphine, triethylphosphine, tri-n-propylphosphine, tri-iso-propylphosphine, tri-n-butylphosphine, tri-iso-butylphosphine, tri-tert-butylphosphine, tricyclopentylphosphine, triallylphosphine, tricyclohexylphosphine, triphenylphosphine, trimethyl phosphite, triethyl phosphite, tri-n-propyl phosphite, tri-iso-propyl phosphite, tri-n-butyl phosphite, tri-isobutyl phosphite, tri-tert-butyl phosphite, tricyclopentyl phosphite, triallyl phosphite, tricyclohexyl phosphite, and triphenyl phosphite.

Other examples of organophosphorus compounds suitable as Lewis bases include phosphinite and phosphonite ligands. Representative examples of phosphinite ligands include methyl diphenylphosphinite, ethyl diphenylphosphinite, isopropyl diphenylphosphinite, and phenyl diphenylphosphinite. Representative examples of phosphonite ligands include diphenyl phenylphosphonite, dimethyl phenylphosphonite, diethyl methylphosphonite, diisopropyl phenylphosphonite, and diethyl phenylphosphonite.

If added, the Lewis base may typically be added in a stoichiometric excess amount, although this is not a requirement.

Exemplary activators include: lithium tetrakis(2-fluorophenyl)borate, sodium tetrakis(2-fluorophenyl)borate, silver tetrakis(2-fluorophenyl)borate, thallium tetrakis(2-fluorophenyl)borate, lithium tetrakis(3-fluorophenyl)borate, sodium tetrakis(3-fluorophenyl)borate, silvertetrakis(3-fluorophenyl)borate, thallium tetrakis(3-fluorophenyl)borate, ferroceniun tetrakis(3-fluorophenyl)borate, ferrocenium tetrakis(pentafluorophenyl)borate, lithium tetrakis(4-fluorophenyl)borate, sodium tetrakis(4-fluorophenyl)borate, silver tetrakis(4-fluorophenyl)borate, thallium tetrakis(4-fluorophenyl)borate, lithium tetrakis(3,5-difluorophenyl)borate, sodium tetrakis(3,5-difluorophenyl)borate, thallium tetrakis(3,5-difluorophenyl)borate, trityl tetrakis(3,5-difluorophenyl)borate, 2,6-dimethylanilinium tetrakis(3,5-difluorophenyl)borate, lithium tetrakis(pentafluorophenyl)borate, lithium (diethyl ether)tetrakis(pentafluorophenyl)borate, lithium (diethyl ether)tetrakis(pentafluorophenyl) borate, lithium tetrakis(2,3,4,5-tetrafluorophenyl)borate, lithium tetrakis(3,4,5,6-tetrafluorophenyl)borate, lithium tetrakis(1,2,2-trifluoroethylenyl)borate, lithium tetrakis(3,4,5-trifluorophenyl)borate, lithium methyltris(perfluorophenyl)borate, lithium phenyltris(perfluorophenyl)borate, lithium tris(isopropanol)tetrakis(pentafluorophenyl)borate, lithium tetrakis(methanol)tetrakis(pentafluorophenyl)borate, silver tetrakis(pentafluorophenyl)borate, tris(toluene)silver tetrakis(pentafluorophenyl)borate, tris(xylene)silver tetrakis(pentafluorophenyl)borate, trityl tetrakis(pentafluorophenyl)borate, trityl tetrakis(4-triisopropylsilyltetrafluorophenyl)borate, trityl tetrakis(4-dimethyl-tert-butylsilyl-tetrafluorophenyl)borate, thallium tetrakis[3,5-bis(trifluoromethyl)phenyl]borate, 2,6-dimethylanilinium tetrakis(pentafluorophenyl)borate, N,N-dimethylanilinium tetrakis(pentafluorophenyl)borate, N,N-dimethylanilinium tetrakis[3,5-bis(trifluoromethyl)phenyl]borate, lithium (triphenylsiloxy)tris(pentafluorophenyl)borate, sodium (triphenylsiloxy)tris(pentafluorophenyl)borate, sodium tetrakis(2,3,4,5-tetrafluorophenyl)borate, sodium tetrakis(3,4,5,6-tetrafluorophenyl)borate, sodium tetrakis(1,2,2-trifluoroethylenyl)borate, sodium tetrakis(3,4,5-trifluorophenyl)borate, sodium methyltris(perfluorophenyl)borate, sodium phenyltris(perfluorophenyl)borate, thallium tetrakis(2,3,4,5-tetrafluorophenyl)borate, thallium tetrakis(3,4,5,6-tetrafluorophenyl)borate, thallium tetrakis(1,2,2-trifluoroethylenyl)borate, thallium tetrakis(3,4,5-trifluorophenyl)borate, sodium methyltris(perfluorophenyl)borate, thallium phenyltris(perfluorophenyl)borate, trityl tetrakis(2,3,4,5-tetrafluorophenyl)borate, trityl tetrakis(3,4,5,6-tetrafluorophenyl)borate, trityl tetrakis(1,2,2-trifluoroethylenyl)borate, trityl tetrakis(3,4,5-trifluorophenyl)borate, trityl methyltris(pentafluorophenyl)borate, trityl phenyltris-(perfluorophenyl)borate, silver tetrakis[3,5-bis(trifluoromethyl)phenyl]borate, silver(toluene) tetrakis[3,5-bis(trifluoromethyl)phenyl]borate, thallium tetrakis[3,5-bis(trifluoromethyl)phenyl]borate, lithium hexyltris(pentafluorophenyl)borate, lithium tniphenylsiloxytris(pentafluorophenyl)borate, lithium (octyloxy)tris(pentafluorophenyl)borate, lithium tetrakis(3,5-bis(trifluoronethyl)phenyl)borate, sodium tetrakis(pentafluorophenyl)borate, trityl tetrakis(pentafluorophenyl)borate, sodium (octyloxy)tris(pentafluorophenyl)borate, sodium tetrakis(3,5-bis(trifluoromethyl)phenyl)borate, potassium tetrakis(pentafluorophenyl)borate, trityl tetrakis(pentafluorophenyl)borate, potassium (octyloxy)tris(pentafluorophenyl)borate, potassium tetrakis(3,5-bis(trifluoromethyl)phenyl)borate, magnesium tetrakis(pentafluorophenyl)borate, magnesium (octyloxy)tris(pentafluorophenyl)borate, magnesium tetrakis(3,5-bis(trifluoro-methyl)phenyl)borate, calcium tetrakis(pentafluorophenyl)borate, calcium (octyloxy)tris(pentafluorophenyl)borate, calcium tetrakis(3,5-bis(trifluoromethyl)phenyl)borate, lithium tetrakis[3,5-bis[1-methoxy-2,2,2-trifluoro-1-trifluoromethyl)ethyl]phenyl]borate, sodium tetrakis[3,5-bis[1-methoxy-2,2,2-trifluoro-1-(trifluoromethyl)ethyl]phenyl]borate, silver tetrakis[3,5-bis[1-methoxy-2,2,2-trifluoro-1-(trifluoromethyl)ethyl]phenyl]borate, thallium tetrakis[3,5-bis[1-methoxy-2,2,2-trifluoro-1-(trifluoromethyl)ethyl]phenyl]borate, lithium tetrakis[3-[1-methoxy-2,2,2-trifluoro-1-(trifluoromethyl)ethyl]-5-(trifluoromethyl)phenyl]borate, sodium tetrakis[3-[1-methoxy-2,2,2-trifluoro-1-(trifluoromethyl)ethyl]-5-(trifluoromethyl)phenyl]borate, silver tetrakis[3-[1-methoxy-2,2,2-trifluoro-1-(trifluoromethyl)ethyl]-5-(trifluoromethyl)phenyl]borate, thallium tetrakis[3-[1-methoxy-2,2,2-trifluoro-1-(trifluoromethyl)ethyl]-5-(trifluoromethyl)phenyl]borate, lithium tetrakis[3-[2,2,2-trifluoro-1-(2,2,2-trifluoroethoxy)-1-(trifluoromethyl)ethyl]-5-(trifluoromethyl)phenyl]borate, sodium tetrakis[3-[2,2,2-trifluoro-1-(2,2,2-trifluoroethoxy)-1-(trifluoromethyl)ethyl]-5-(trifluoromethyl)phenyl]borate, silver tetrakis [3-[2,2,2-trifluoro-1-(2,2,2-trifluoroethoxy)-1-(trifluoromethyl)ethyl]-5-(trifluoromethyl)phenyl]borate, thallium tetrakis[3-[2,2,2-trifluoro-1-(2,2,2-trifluoroethoxy)-1-(trifluoromethyl)ethyl]-5-(trifluoromethyl)phenyl]borate, trimethylsilylium tetrakis(pentafluorophenyl)borate, trimethylsilylium etherate tetrakis-(pentafluorophenyl)borate, triethylsilylium tetrakis(pentafluorophenyl)borate, triphenylsilylium tetrakis(pentafluorophenyl)borate, tris(mesityl)silylium tetrakis(pentafluorophenyl)borate, tribenzylsilylium tetrakis(pentafluorophenyl)borate, trimethylsilylium methyltris(pentafluorophenyl)borate, triethylsilylium methyltris(pentafluorophenyl)borate, triphenylsilylium methyltris(pentafluorophenyl)borate, tribenzylsilylium methyltris(pentafluorophenyl)borate, trimethylsilylium tetrakis(2,3,4,5-tetrafluorophenyl)borate, triethylsilylium tetrakis(2,3,4,5-tetrafluorophenyl)borate, triphenylsilylium tetrakis(2,3,4,5-tetrafluorophenyl)borate, tribenzylsilylium tetrakis(2,3,4,5-tetrafluorophenyl)borate, trimethylsilylium tetrakis(2,3,4,5-tetrafluorophenyl) borate, triphenylsilylium tetrakis(2,3,4,5-tetra-fluorophenyl)borate, trimethylsilylium tetrakis(3,4,5-trifluorophenyl)borate, tribenzylsilylium tetrakis(3,4,5-trifluorophenyl)aluminate, triphenylsilylium methyltris(3,4,5-trifluorophenyl) aluminate, triethylsilylium tetrakis(1,2,2-trifluoroethenyl)borate, tricyclohexylsilylium tetrakis(2,3,4,5-tetrafluorophenyl)borate, dimethyloctadecylsilylium tetrakis(pentafluorophenyl)borate, tris(trimethylsilyl)silylium methyltris(2,3,4,5-tetrafluorophenyl)borate, 2,2′-dimethyl-1,1′-binaphthylmethylsilylium tetrakis(pentafluorophenyl)borate, 2,2′-dimethyl-1,1′-binaphthylmethylsilylium tetrakis(3,5-bis(trifluoromethyl)phenyl)borate, lithium tetrakis(pentafluorophenyl)aluminate, trityl tetrakis(pentafluorophenyl)aluminate, trityl (perfluorobiphenyl)fluoroaluminate, lithium (octyloxy)tris(pentafluorophenyl)aluminate, lithium tetrakis(3,5-bis(trifluoromethyl)phenyl)aluminate, sodium tetrakis(pentafluorophenyl)aluminate, trityl tetrakis(pentafluorophenyl)aluminate, sodium (octyloxy)tris(pentafluorophenyl)aluminate, sodium tetrakis(3,5-bis(trifluoromethyl)phenyl)aluminate, potassium tetrakis(pentafluorophenyl)aluminate, trityl tetrakis(pentafluorophenyl)aluminate, potassium (octyloxy)tris(pentafluorophenyl)aluminate, potassium tetrakis(3,5-bis(trifluoromethyl)-phenyl)aluminate, magnesium tetrakis(pentafluorophenyl)aluminate, magnesium (octyloxy)tris-(pentafluorophenyl)aluminate, magnesium tetrakis(3,5-bis(trifluoromethyl)phenyl)aluminate, calcium tetrakis(pentafluorophenyl)aluminate, calcium (octyloxy)tris(pentafluorophenyl)aluminate, calcium tetrakis(3,5-bis(trifluoro-methyl)phenyl)aluminate, LiB(OC(CF₃)₃)₄, LiB(OC(CF₃)₂(CH₃))₄, LiB(OC(CF₃)₂H)₄, LiB(OC(CF₃)(CH₃)H)₄, Tl(OC(CF₃)₃)₄, TlB(OC(CF₃)₂H)₄, TlB(OC(CF₃)(CH₃)H)₄, TlB(OC(CF₃)₂(CH₃))₄, (Ph₃C)B(OC(CF₃)₃)₄, (Ph₃C)B(OC(CF₃)₂(CH₃))₄, (Ph₃C)B(OC(CF₃)₂H)₄, (Ph₃C)B(OC(CF₃)(CH₃)H)₄, AgB(OC(CF₃))₄, AgB(OC(CF₃)₂₁)₄, AgB(OC(CF₃)(CH₃)H)₄, LiB(O₂C₆F₄)₂, TlB(O₂C₆F₄)₂, Ag(toluene)₂B(O₂C₆F₄)₂, Ph₃CB(O₂C₆F₄)₂, LiB(OCH(CF₃)₂)₄, [Li(HOCH₃)₄]B(O₂C6Cl₄)₂, [Li(HOCH₃)₄]B(O₂C₆F₄)₂, [Ag(toluene)₂]B(O₂C6Cl₄)₂, LiB(O₂C₆Cl₄)₂, (LiAl(OC(CF₃)₂Ph)₄), (TlAl(OC(CF₃)₂Ph)₄), (AgAl(OC(CF₃)₂Ph)₄), (Ph₃CAl(OC(CF₃)₂Ph)₄, (LiAl(OC(CF₃)₂C₆H₄CH₃)₄), (TlAl(OC(CF₃)₂C₆H₄CH₃)₄), (AgAl(OC(CF₃)₂C₆H₄CH₃)₄), (Ph₃CAl(OC(CF₃)₂C₆H₄CH₃)₄), LiAl(OC(CF₃))₄, TlAl(OC(CF₃)₃)₄, AgAl(OC(CF₃)₃)₄, Ph₃CAl(OC(CF₃)₃)₄, LiAl(OC(CF₃)(CH₃)H)₄, TlAl(OC(CF₃)(CH₃)H)₄, AgAl(OC(CF₃)(CH₃)H)₄, Ph₃CAl(OC(CF₃)(Cl₃)H)₄, LiAl(OC(CF₃)₂H)₄, TlAl(OC(CF₃)₂H)₄, AgAl(OC(CF₃)₂H)₄, Ph₃CAl(OC(CF₃)₂H)₄, LiAl(OC(CF₃)₂C₆H₄-4-i-Pr)₄, TlAl(OC(CF₃)₂C₆H₄-4-i-Pr)₄, AgAl(OC(CF₃)₂C₆H₄-i-Pr)₄, Ph₃CAl(OC(CF₃)₂C₆H₄-4-i-Pr)₄, LiAl(OC(CF₃)₂C₆H₄-t-butyl)₄, TlAl(OC(CF₃)₂C₆H₄-t-butyl)₄, AgAl(OC(CF₃)₂C₆H₄-4-t-butyl)₄, LiAl(OC(CF₃)₂C₆H₄-4-SiMe₃)₄. TlAl(OC(CF₃)₂C₆H₄-4-SiMe₃)₄, AgAl(OC(CF₃)₂C₆H₄-4-SiMe₃)₄, Ph₃CAl(OC(CF₃)₂C₆H₄-4-SiMe₃)₄, LiAl(OC(CF₃)₂C₆H₄-4-Si-i-Pr₃)₄, TiAl(OC(CF₃)₂C₆H₄-4-Si-i-Pr₃)₄, AgAl(OC(CF₃)₂C₆H₄-4-Si-i-Pr₃)₄, Ph₃CAl(OC(CF₃)₂C₆H₄-4-Si-i-Pr₃)₄, LiAl(OC(CF₃)₂C₆H₂-2,6-(CF₃)₂-4-Si-i-Pr₃)₄, TlAl(OC(CF₃)₂C₆H₂-2,6-(CF₃)₂-4-Si-i-Pr₃)₄, AgAl(OC(CF₃)₂C₆H₂-2,6-(CF₃)₂-4-Si-i-Pr₃)₄, Ph₃CAl(OC(CF₃)₂C₆H₂-2,6-(CF₃)₂-4-Si-i-Pr₃)₄, LiAl(OC(CF₃)₂C₆H₃-3,5-(CF₃)₂)₄, TlAl(OC(CF₃)₂C₆H₃-3,5-(CF₃)₂)₄, AgAl(OC(CF₃)₂C₆H₃-3,5-(CF₃)₂)₄, Ph₃CAl(OC(CF₃)₂C₆H₃-3,5-(CF₃)₂)₄, LiAl(OC(CF₃)₂C₆H₂-2,4,6-(CF₃))₄, TlAl(OC(CF₃)₂C₆H₂-2,4,6-(CF₃)₃)₄, AgAl(OC(CF₃)₂C₆H₂-2,4,6-(CF₃)₃)₄-Ph₃CAl(OC(CF₃)₂C₆H₂-2,4,6-(CF₃L)₄, LiAl(OC(CF₃)₂C₆F₅)₄, TlAl(OC(CF₃)₂C₆F₅)₄, AgAl(OC(CF₃)₂C₆F₅)₄, Ph₃CAl(OC(CF₃)₂C₆F₅)₄, [1,4-dihydro-4-methyl-1-(pentafluorophenyl)]-2-borinyl lithium, [1,4-dihydro-4-methyl-1-(pentafluorophenyl)]-2-borinyl triphenylmethylium, 4-(1,1-dimethyl)-1,2-dihydro-1-(pentafluorophenyl)-2-borinyllithium, 4-(1,1-dimethyl)-1,2-dihydro-1-(pentafluorophenyl)-2-borinyltriphenylmethylium, 1-fluoro-1,2-dihydro-4-(pentafluorophenyl)-2-borinyllithium, 1-fluoro-1,2-dihydro-4-pentafluorophenyl)-2-borinyl triphenylmethylium, 1-[3,5-bis(trifluoromethyl)phenyl]-1,2-dihydro-4-(pentafluorophenyl)-2-borinyl lithium, 1-[3,5-bis(trifluoromethyl)phenyl]-1,2-dihydro-4-(pentafluorophenyl)-2-borinyl triphenylmethylium, LiBF₄, NaBF₄, KBF₄, AgBF₄, [Ph₃C]BF₄, TlBF₄, Mg(BF₄)₂, Ca(BF₄)₂, LiPF₆, NaPF₆, KPF₆, [Ph₃C]PF₆, TlPF₆, Mg(PF₆)₂, Ca(PF₆)₂, LiSbF₆, NaSbF₆, KSbF₆, [Ph₃C]SbF₆, TlSbF₆, Mg(SbF₆)₂, and Ca(SbF₆)₂.

Typically, the molar ratio of activator to pro-catalyst is in the range of 10:1 to 1:10, preferably 10:1 to 1:1, although other ratios may also be used.

Many useful addition polymerization catalysts and pro-catalyst/activator combinations are known and are disclosed, for example, in col. 8, line 28 to col. 9 line 56 of U.S. Pat. No. 3,330,815 (McKeon et al.); col. 3, line 9 to col. 17, line 16 of U.S. Pat. No. 6,455,650 B1 (Lipian et al.); col. 3, line 18 to col. 31, line 53 of U.S. Pat. No. 6,825,307 (Goodall); col. 3, line 31 to col. 17, line 16 of U.S. Pat. No. 6,903,171 B2 (Rhodes et al.); and col. 16, line 32 to col. 28, line 31 of U.S. Pat. No. 7,759,439 B2 (Rhodes et al.); col. 20, line 28 to col. 21, line 30 in U.S. Pat. No. 10,266,720 (Burgoon et al.); and paragraphs [0015] to [0075] of U.S. Pat. Appl. Publ. 2005/0187398 A1 (Bell et al.), the disclosures of which are incorporated herein by reference. Another category of catalysts involves pro-catalyst complexes of early or late metals that do not initially have alkyl/allyl ligands but are alkylated by a cocatalyst such as, for example, methylaluminoxane.

Details concerning certain addition polymerization catalysts are also reported by M. V. Bermeshev and P. P. Chapala in “Addition polymerization of functionalized norbornenes as a powerful tool for assembling molecular moieties of new polymers with versatile properties”, Progress in Polymer Science (2018), 84, pp. 1-46.

The addition polymerization catalyst may be included in any effective amount to cause at least partial polymerization of a polymerizable composition, optionally with heating. Typically, the amount of addition polymerization catalyst can vary from about 0.0001 part by weight to about 20 parts by weight based on 100 parts by weight of the addition polymerizable compounds that are present in the polymerizable composition, preferably from about 0.01 part by weight to about 5 parts by weight per 100 parts by weight of the addition polymerizable compounds that are present in the polymerizable composition; however, this is not a requirement.

In some cases, the copolymer is a reaction product of a polymerizable composition comprising isomer A and isomer B or consisting essentially of isomer A and isomer B. By “consisting essentially of” is meant that that copolymer is formed of 95 wt. % to 100 wt. % of isomer A and isomer B. A polymerizable composition consisting essentially of isomer A and isomer B my contain up to 5 wt. % other components, such as additives, impurities, etc.

In some cases, the copolymer is a reaction product of a polymerizable composition comprising isomer A and isomer B and at least one additional monomer, such as a norbornene-type monomer.

The at least one additional monomer (e.g., a combination of all additional monomers) may be present in the polymerizable composition in an amount of 5 percent by mole (mol %) or greater, based on the total moles of a combination of isomer A, isomer B, and the one or more additional monomers of the polymerizable composition, 6 mol %, 7 mol %, 8 mol %, 9 mol %, 10 mol %, 12 mol %, 15 mol %, 17 mol %, 20 mol %, 22 mol %, 25 mol %, 27 mol %, 30 mol %, 32 mol %, 35 mol %, 37 mol %, 40 mol %, 42 mol %, 45 mol %, 47 mol %, 50 mol %, 52 mol %, 55 mol %, 57 mol %, 60 mol %, 62 mol %, 65 mol %, 67 mol %, or 70 mol % or greater; and 95 mol % or less, 92 mol %, 90 mol %, 87 mol %, 85 mol %, 82 mol %, 80 mol %, 77 mol %, 75 mol %, 72 mol %, 70 mol %, 67 mol %, 65 mol %, 62 mol %, 60 mol %, 57 mol %, 55 mol %, 52 mol %, 50 mol %, 47 mol %, 45 mol %, 42 mol %, 40 mol %, 37 mol %, 35 mol %, 32 mol %, 30 mol %, 27 mol %, 25 mol %, 22 mol %, or 20 mol % or less, based on the total moles of polymerizable components of the polymerizable composition. In certain cases, the additional monomer(s) may be present in the polymerizable composition in an amount of 40 mol % to 60 mol %.

The isomer A may be present in the polymerizable composition in an amount of 0.5 mol % or greater, based on the total moles of a combination of isomer A, isomer B, and the one or more additional monomers of the polymerizable composition, 1 mol %, 1.5 mol %, 2 mol %, 2.5 mol %, 3 mol %, 3.5 mol %, 4 mol %, 4.5 mol %, 5 mol %, 6 mol %, 7 mol %, 8 mol %, 9 mol %, 10 mol %, 11 mol %, 12 mol %, 13 mol %, 14 mol %, 15 mol %, 17 mol %, 20 mol %, 22 mol %, 25 mol %, 27 mol %, 30 mol %, 32 mol %, 35 mol %, 37 mol %, 40 mol %, 42 mol %, 45 mol %, 47 mol %, or 50 mol %; and 75 mol % or less, 72 mol %, 70 mol %, 67 mol %, 65 mol %, 62 mol %, 60 mol %, 57 mol %, 55 mol %, 52 mol %, 50 mol %, 47 mol %, 45 mol %, 42 mol %, 40 mol %, 37 mol %, 35 mol %, 32 mol %, 30 mol %, 27 mol %, 25 mol %, 22 mol %, or 20 mol % or less, based on the total moles of polymerizable components of the polymerizable composition.

The isomer B may be present in the polymerizable composition in an amount of 1 mol % or greater, based on the total moles of a combination of isomer A, isomer B, and the one or more additional monomers of the polymerizable composition, 1.5 mol %, 2 mol %, 2.5 mol %, 3 mol %, 3.5 mol %, 4 mol %, 4.5 mol %, 5 mol %, 6 mol %, 7 mol %, 8 mol %, 9 mol %, 10 mol %, 11 mol %, 12 mol %, 13 mol %, 14 mol %, 15 mol %, 17 mol %, 20 mol %, 22 mol %, 25 mol %, 27 mol %, 30 mol %, 32 mol %, 35 mol %, 37 mol %, 40 mol %, 42 mol %, 45 mol %, 47 mol %, 50 mol %; 52 mol %, 55 mol %, 57 mol %, 60 mol %, 62 mol %, 65 mol %, 67 mol %, or 70 mol % or greater; and 90 mol % or less, 87 mol %, 85 mol %, 82 mol %, 80 mol %, 77 mol %, 75 mol % or less, 72 mol %, 70 mol %, 67 mol %, 65 mol %, 62 mol %, 60 mol %, 57 mol %, 55 mol %, 52 mol %, 50 mol %, 47 mol %, 45 mol %, 42 mol %, 40 mol %, 37 mol %, 35 mol %, 32 mol %, 30 mol %, 27 mol %, 25 mol %, 22 mol %, or 20 mol % or less, based on the total moles of polymerizable components of the polymerizable composition.

Exemplary suitable norbornene-type monomers include the addition polymerizable cycloolefins mentioned above. In some cases, a suitable monomer includes monomers of formula (C):

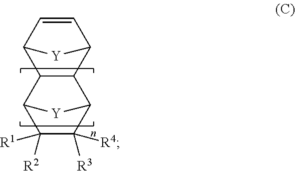

wherein each of R¹, R², R³, and R⁴ is independently selected from H, a linear or branched C1 to C20 hydrocarbyl; a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

A C1 to C20 group such as those mentioned throughout the present disclosure has 1 or more carbon atoms, 2, 3, 4, 5, 6, 7, 8, 9, 10, 11, or 12 or more carbon atoms, and 20 or fewer carbon atoms, 19, 18, 17, 16, 15, 14, 13, 12, 10, 9, 8, 7, 6, 5, or 4 or fewer carbon atoms.

In select embodiments, in a monomer of formula (C) as depicted above, each of R¹, R², R³, and R⁴ is H; n is 0; and Y is —CH₂—. In select embodiments, in a monomer of formula (C) as depicted above, one of R¹, R², R³, and R⁴ is a C6 alkyl group; each of the rest of R¹, R², R, and R⁴ is H; n is 0; and Y is —CH₂—. Stated another way, that copolymer comprises a reaction product of a polymerizable composition comprising isomer A, isomer B, and hexylnorbornene.

Optionally, a copolymer comprises at least two monomers of formula (C) that are different from each other. In one embodiment, a copolymer comprises at least two monomers of formula (C) that are different from each other, wherein in a first of the monomers of formula (C) each of R¹, R², R³, and R⁴ is H; n is 0; and Y is —CH₂— and in a second of the monomers of formula (C) one of R¹, R², R, and R⁴ is a C6 alkyl group; each of the rest of R¹, R², R, and R⁴ is H; n is 0; and Y is —CH₂—. Stated another way, that copolymer comprises a reaction product of a polymerizable composition comprising isomer A, isomer B, norbornene, and hexylnorbornene.

Suitable solvents for preparing copolymers described herein include ethers such as diethyl ether, ethyl propyl ether, dipropyl ether, methyl t-butyl ether, di-t-butyl ether, glyme (dimethoxyethane), diglyme, diethylene glycol dimethyl ether; cyclic ethers such as tetrahydrofuran and dioxane; alkanes; cycloalkanes; aromatic hydrocarbon solvents such as benzene, toluene, o-xylene, m-xylene, p-xylene; halogenated hydrocarbon solvents; acetonitrile; lactones such as butyrolactone, and valerolactones; ketones such as acetone, methyl ethyl ketone, methyl isobutyl ketone, cyclopentanone, and cyclohexanone; sulfones such as tetramethylene sulfone, 3-methylsulfolane, 2,4-dimethylsulfolane, butadiene sulfone, methyl sulfone, ethyl sulfone, propyl sulfone, butyl sulfone, methyl vinyl sulfone, 2-(methylsulfonyl) ethanol, and 2,2′-sulfonyldiethanol; sulfoxides such as dimethyl sulfoxide; cyclic carbonates such as propylene carbonate, ethylene carbonate and vinylene carbonate; carboxylic acid esters such as ethyl acetate, Methyl Cellosolve™ and methyl formate; alcohols such as ethanol, methanol, and propanol; and other solvents such as methylene chloride, nitromethane, acetonitrile, glycol sulfite and 1,2-dimethoxyethane (glyme), and combinations of such solvents.

Molecular weight of a copolymer may be controlled through the inclusion of one or more chain transfer agents or chain retarding agents, in a polymerizable composition. For instance, a suitable chain transfer agent includes monofunctional terminal chain transfer agents such as formic acid and primary alkenes such as 1-octene, alcohols, plus others such as are known in the art.

Copolymers according to the present disclosure can be prepared, for example, by reacting a polymerizable composition for sufficient time and at sufficient temperature to result in at least partial curing, preferably substantially full polymerization, of the polymerizable components. The heating temperature will depend on the specific polymerizable composition and may be at room temperature (i.e., 20-25° C.), at least 30° C., at least 40° C., at least 50° C., at least 75° C., at least 100° C., or at least 150° C., for example. Typically, the reaction time for complete cure will be from several minutes to 5 days, preferably from 30 minutes to 3 days, and most preferably from 1 to 24 hours. In certain cases, a copolymer according to the present disclosure is provided in a form of a film.

In a third aspect, another copolymer is provided. The copolymer comprises the following divalent monomer units:

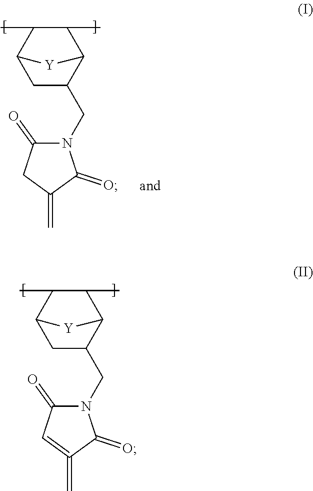

wherein each Y is independently selected from —CH₂—, —CH₂CH₂—, and O.

In a fourth aspect, a further copolymer is provided. The copolymer comprises the following divalent monomer units:

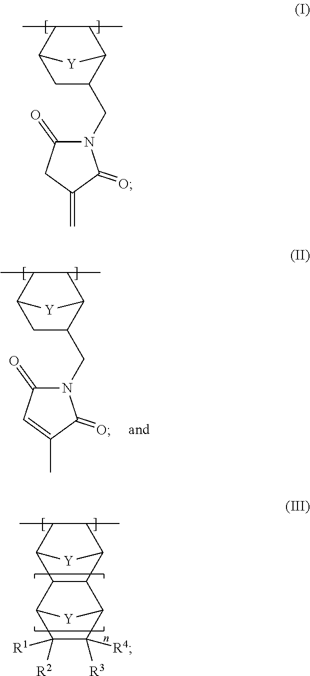

wherein each of R¹, R², R³, and R⁴ is independently selected from H, a linear or branched C1 to C20 hydrocarbyl, a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

The below disclosure relates to both the third aspect and the fourth aspect.

In select embodiments, in a divalent unit of formula (III), at least one of R¹, R², R³, and R⁴ is a C₁ to C₇ linear alkyl, vinyl, or a C1 to C20 carbosilane. In select embodiments, in a divalent unit of formula (III), each of R¹, R², R³, and R⁴ is H; n is 0; and Y is —CH₂—. In select embodiments, R¹ and R² taken together or R³ and R⁴ taken together form a C1 to C20 hydrocarbylidene group, e.g., the hydrocarbylidene group may be ethylidene or another alkylidene. In select embodiments, in a divalent unit of formula (III), one of R¹, R², R³, and R⁴ is a C6 alkyl group; each of the rest of R¹, R², R³, and R⁴ is H; n is 0; and Y is —CH₂—. Stated another way, that copolymer comprises a reaction product of a polymerizable composition comprising isomer A, isomer B, and hexylnorbornene.

Optionally, the copolymer comprises at least two divalent units of formula (III) that are different from each other. For instance, in one such copolymer, in a first of the divalent units (III), each of R¹, R², R³, and R⁴ is H; n is 0; and Y is —CH₂—; and in a second of the divalent units (III), one of R¹, R², R³, and R⁴ is a C6 alkyl group and each of the rest of R¹, R², R³, and R⁴ is H; n is 0; and Y is —CH₂—. Stated another way, that copolymer comprises a reaction product of a polymerizable composition comprising isomer A, isomer B, norbornene, and hexylnorbornene.

The copolymers of the third aspect and the fourth aspect may be prepared as described above in detail with respect to the first and second aspects. In certain cases, a copolymer according to the third aspect or the fourth aspect is provided in a form of a film.

In a fifth aspect, a composition is provided. The composition comprises a solvent and the copolymer of any of the second through fourth aspects described in detail above.

In a sixth aspect, a film is provided. The film comprises a crosslinked reaction product of the copolymer of any of the second through fourth aspects described in detail above.

The below disclosure relates to both the fifth aspect and the sixth aspect.

Solvent in the composition may be used to dissolve a copolymer according to the present disclosure (i.e., any of the copolymers of the second through fourth aspects). Use of a solvent can allow for manipulation of a shape of the copolymer, e.g., by making it possible to form a film out of the copolymer upon removal of the solvent. Exemplary solvents include, for example, the solvents listed above with respect to the first and second aspects.

The composition may also include one or more additives. Examples of suitable additives include sensitizers, colorants (e.g., pigments and/or dyes), initiators, antioxidants, inhibitors, thermal degradation stabilizers, light stabilizers (e.g., UV stabilizers), inert fillers, adhesion control agents, and other additives known to those skilled in the art. They also can be substantially unreactive, such as fillers, both inorganic and organic, reinforcing agents, solid fillers, stabilizers, and combinations thereof. The additives may be added in amounts sufficient to obtain the desired properties for the cured composition being produced. The desired properties are largely dictated by the intended application of the resultant copolymer or film. In select embodiments, the composition further comprises at least one of a sensitizer, an initiator, a filler (e.g., silica), an antioxidant, an inhibitor, a stabilizer, a colorant, or an adhesion control agent (e.g., silanes, zirconates, and/or titanates).

When an initiator is present, optionally the initiator comprises at least one of a thermal initiator or a photoinitiator.

Exemplary thermal curing initiators for use herein may be selected from the group consisting of rapid-reacting thermally-initiated curing initiators, latent thermally-initiated curing initiators, and any combinations or mixtures thereof. Suitable thermal initiators include for instance and without limitation, peroxides such as benzoyl peroxide, dibenzoyl peroxide, dilauryl peroxide, cyclohexane peroxide, dicumyl peroxide, methyl ethyl ketone peroxide, hydroperoxides, e.g., tert-butyl hydroperoxide and cumene hydroperoxide, dicyclohexyl peroxydicarbonate, and t-butyl perbenzoate, and diazo compounds such as 2,2,-azo-bis(isobutyronitrile). Examples of commercially available thermal initiators include: initiators available from Chemours Co. (Wilmington, DE) under the VAZO trade designation including VAZO 67 (2,2′-azo-bis(2-methybutyronitrile)), VAZO 64 (2,2′-azo-bis(isobutyronitrile)), and VAZO 52 (2,2′-azo-bis(2,2-dimethyvaleronitrile)), and LUPEROX A98 from Arkema (King of Prussia, PA). Crosslinking of the copolymer of the film is effected by exposing the film containing a thermal initiator to a sufficient elevated temperature to activate the thermal initiator, as known to the skilled practitioner.

In some embodiments, the initiator comprises a photoinitiator. Photoinitiators are used with actinic radiation, often with ultraviolet (UV) light, although other light sources could be used with the appropriate choice of initiator, such as visible light initiators, infrared light initiators, and the like. Typically, UV photoinitiators are used. Crosslinking of the copolymer of the film is effected by exposing the film containing a photoinitiator to the light source to activate the photoinitiator, as known to the skilled practitioner.

Photoinitiators typically comprise photoinitiator groups selected from acyl phosphine oxide, alkyl amine acetophenone, benzil ketal, hydroxy-acetophenone, organic or inorganic peroxide, a persulfate, titanocene complex, or azo. When the initiator groups include a persulfate, tetramethylethylenediamine may also be included as a curing accelerator.

Exemplary photoinitiators include benzoin and its derivatives such as alpha-methylbenzoin; alpha-phenylbenzoin; alpha-allylbenzoin; alpha benzylbenzoin; benzoin ethers such as benzil dimethyl ketal (e.g., “OMNIRAD BDK” from IGM Resins USA Inc., Charlotte, NC), benzoin methyl ether, benzoin ethyl ether, benzoin n-butyl ether; acetophenone and its derivatives such as 2-hydroxy-2-methyl-1-phenyl-1-propanone (e.g., available under the trade designation OMNIRAD 1173 from IGM Resins USA Inc., Charlotte, NC) and 1-hydroxycyclohexyl phenyl ketone (e.g., available under the trade designation OMNIRAD 184 from IGM Resins USA Inc., Charlotte, NC); 2-methyl-1-[4-(methylthio)phenyl]-2-(4-morpholinyl)-1-propanone (e.g., available under the trade designation OMNIRAD 907 from IGM Resins USA Inc., Charlotte, NC); 2-benzyl-2-(dimethylamino)-1-[4-(4-morpholinyl)phenyl]-1-butanone (e.g., available under the trade designation OMNIRAD 369 from IGM Resins USA Inc., Charlotte, NC); polyethylene glycol di(beta-4-[4-(2-dimethylamino-2-benzyl)butanoylphenyl]piperazine)propionate (available under the trade designation OMNIPOL 910 from IGM Resins USA Inc., Charlotte, NC); and phosphine oxide derivatives such as ethyl-2,4,6-trimethylbenzoylphenyl phosphinate (e.g. available under the trade designation TPO-L from IGM Resins USA Inc., Charlotte, NC), and bis-(2,4,6-trimethylbenzoyl)phenylphosphine oxide (e.g., available under the trade designation OMNIRAD 819 from IGM Resins USA Inc., Charlotte, NC).

Other useful photoinitiators include, for example, pivaloin ethyl ether, anisoin ethyl ether, anthraquinones (e.g., anthraquinone, 2-ethylanthraquinone, 1-chloroanthraquinone, 1,4-dimethylanthraquinone, 1-methoxyanthraquinone, or benzanthraquinone), halomethyltriazines, benzophenone and its derivatives, iodonium salts, titanium complexes such as bis(q-5-2,4-cyclopentadien-1-yl)-bis[2,6-difluoro-3-(1H-pyrrol-1-yl)-phenyl]titanium (e.g., available under the trade designation CGI 784DC from BASF, Florham Park, NJ); halomethyl-nitrobenzenes (e.g., 4-bromomethylnitrobenzene), and combinations of photoinitiators where one component is a mono- or bis-acylphosphine oxide (e.g., available under the trade designations IRGACURE 1700, IRGACURE 1800, and IRGACURE 1850 from BASF, Florham Park, NJ, and under the trade designation OMNIRAD 4265 from IGM Resins USA Inc., Charlotte, NC).

In some embodiments the composition may include at least one filler. In some embodiments the total amount of filler is at most 50 wt. %, preferably at most 30 wt. %, and more preferably at most 10 wt. % filler. Fillers may be selected from one or more of a wide variety of materials, as known in the art, and include organic and inorganic filler. Inorganic filler particles include silica, submicron silica, zirconia, submicron zirconia, and non-vitreous microparticles of the type described in U.S. Pat. No. 4,503,169 (Randklev).

Filler components include nanosized silica particles, nanosized metal oxide particles, and combinations thereof. Nanofillers are also described in U.S. Pat. No. 7,090,721 (Craig et al.), U.S. Pat. No. 7,090,722 (Budd et al.), U.S. Pat. No. 7,156,911 (Kangas et al.), and U.S. Pat. No. 7,649,029 (Kolb et al.).

In some embodiments the filler may be surface modified. A variety of conventional methods are available for modifying the surface of nanoparticles including, e.g., adding a surface-modifying agent to nanoparticles (e.g., in the form of a powder or a colloidal dispersion) and allowing the surface-modifying agent to react with the nanoparticles. Other useful surface-modification processes are described in, e.g., U.S. Pat. No. 2,801,185 (Iler), 4,522,958 (Das et al.) and 6,586,483 (Kolb et al.), each incorporated herein by reference.

### EXEMPLARY EMBODIMENTS

In a first embodiment, the present disclosure provides a composition. The composition comprises a mixture of isomers comprising at least 10% by weight of isomer A and up to 90% by weight of isomer B. Isomer A has the following formula:

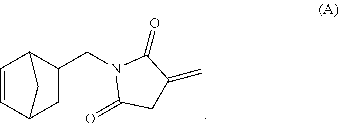 [SMILES: C=C1CC(=O)N(CC2CC3C=CC2C3)C1=O]

Isomer B has the following formula:

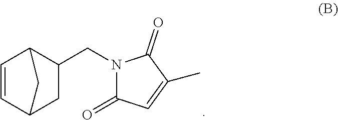 [SMILES: CC1=CC(=O)N(CC2CC3C=CC2C3)C1=O]

In a second embodiment, the present disclosure provides a composition according to the first embodiment, wherein the isomer A is present in an amount of 10% by weight (wt. %) to 75 wt. % of the mixture of isomers and the isomer B is present in an amount of 25 wt. % to 90 wt. % of the mixture of isomers.

In a third embodiment, the present disclosure provides a composition according to the first embodiment or the second embodiment, wherein the isomer A and the isomer B together comprise 95 wt. % to 100 wt. % of the total composition.

In a fourth embodiment, the present disclosure provides a copolymer. The copolymer comprises the following divalent units:

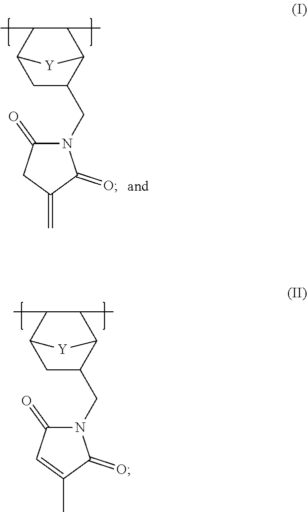

wherein each Y is independently selected from —CH₂—, —CH₂CH₂—, and O.

In a fifth embodiment, the present disclosure provides a copolymer. The copolymer comprises the following divalent units:

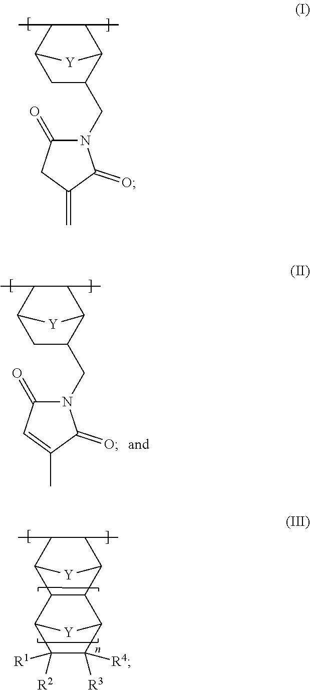

wherein each of R¹, R², R³, and R⁴ is independently selected from H, a linear or branched C1 to C20 hydrocarbyl, a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

In a sixth embodiment, the present disclosure provides a copolymer according to the fifth embodiment, wherein in divalent unit of formula (III), at least one of R¹, R², R³, and R⁴ is a C₁ to C₇ linear alkyl, vinyl, or a C1 to C20 carbosilane.

In a seventh embodiment, the present disclosure provides a copolymer according to the fifth embodiment, wherein in divalent unit of formula (III), each of R¹, R², R³, and R⁴ is H; n is 0; and Y is —CH₂—.

In an eighth embodiment, the present disclosure provides a copolymer according to the fifth embodiment, wherein in divalent unit of formula (III), one of R¹, R², R, and R⁴ is a C6 alkyl group; each of the rest of R¹, R², R³, and R⁴ is H; n is 0; and Y is —CH₂—.

In a ninth embodiment, the present disclosure provides a copolymer according to the fifth embodiment, comprising at least two divalent units of formula (III) that are different from each other.

In a tenth embodiment, the present disclosure provides a copolymer according to the ninth embodiment, wherein in a first of the divalent units of formula (III), each of R¹, R², R³, and R⁴ is H; n is 0; and Y is —CH₂—; and in a second of the divalent units of formula (III), one of R¹, R², R³, and R⁴ is a C6 alkyl group and each of the rest of R¹, R², R³, and R⁴ is H; n is 0; and Y is —CH₂—.

In an eleventh embodiment, the present disclosure provides a copolymer. The copolymer is a reaction product of a polymerizable composition comprising:

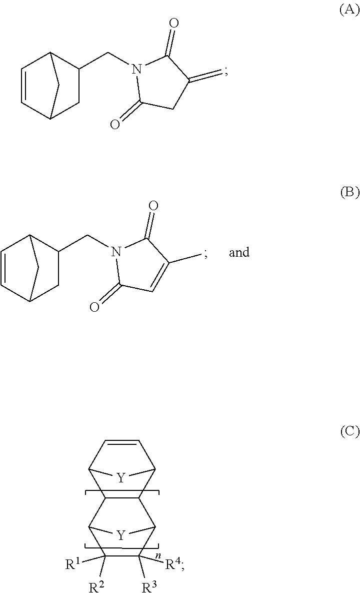

wherein each of R¹, R², R³, and R⁴ is independently selected from H, a linear or branched C1 to C20 hydrocarbyl; a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

In a twelfth embodiment, the present disclosure provides a copolymer according to the eleventh embodiment, wherein the polymerizable composition further comprises a chain transfer agent.

In a thirteenth embodiment, the present disclosure provides a copolymer according to the eleventh embodiment or the twelfth embodiment, comprising at least two monomers of formula (C) that are different from each other, wherein in a first of the monomers of formula (C), each of R¹, R², R³, and R⁴ is H; n is 0; and Y is —CH₂— and in a second of the monomers of formula (C), one of R¹, R², R³, and R⁴ is a C6 alkyl group; each of the rest of R¹, R², R³, and R⁴ is H; n is 0; and Y is —CH₂—.

In a fourteenth embodiment, the present disclosure provides a copolymer according to any of the fourth through thirteenth embodiments, wherein the copolymer is in a form of a film.

In a fifteenth embodiment, the present disclosure provides a composition. The composition comprises a copolymer according to any of the fourth through fourteenth embodiments and a solvent.

In a sixteenth embodiment, the present disclosure provides a composition according to the fifteenth embodiment, further comprising at least one of a sensitizer, an initiator, a filler, an antioxidant, an inhibitor, a stabilizer, a colorant, or an adhesion control agent.

In a seventeenth embodiment, the present disclosure provides a composition according to the sixteenth embodiment, wherein the initiator comprises at least one of a thermal initiator or a photoinitiator.

In an eighteenth embodiment, the present disclosure provides a film. The film comprises a crosslinked reaction product of a copolymer according to any of the fourth through fourteenth embodiments.

Advantages and embodiments of this invention are further illustrated by the following examples, but the particular materials and amounts thereof recited in these examples, as well as other conditions and details, should not be construed to unduly limit this invention.

### EXAMPLES

Unless otherwise noted or apparent from the context, all parts, percentages, ratios, etc. in the Examples and the rest of the specification are by weight. Table 1 (below) lists materials used in the examples and their sources.

TABLE 1
Table of materials, abbreviations, and sources.

| Material | Description |
|---|---|
| Hexylnorbornene, HNB | Synthesized as described in Designed |
| | Monomers and Polymers, Vol. 8, No. |
| | 3, pp. 237-248 (2005). |
| 5-norbornene-2-methylamine | Synthesized as described |
| | in Tetrahedron Letters, 2008, 49(29- |
| | 30), p. 4553-4555 |
| Norbornene, NB | Alfa Aesar (Ward Hill, MA, USA) |
| Itaconic anhydride | TCI Chemicals (Tokyo, Japan) |
| Citraconic anhydride | TCI Chemicals (Tokyo, Japan) |
| Toluene | EMD Millipore (Burlington, MA, |
| | USA) |
| Magnesium sulfate, MgSO₄ | EMD Millipore (Burlington, MA, |
| | USA) |
| Potassium hydroxide, KOH | VWR International (Radnor, PA, |
| | USA) |
| Potassium bicarbonate, KHCO₃ | EM Science (Gibbstown, NJ, USA) |
| Potassium chloride, KCl | VWR International (Radnor, PA, |
| | USA) |
| 1-Octene | Alfa Aesar (Ward Hill, MA, USA) |
| Tricyclohexylphosphine, PCy₃ | Alfa Aesar (Ward Hill, MA, USA) |
| Sodium tetrakis[3,5- | SynQuest Laboratories |
| bis(trifluoromethyl)phenyl]borate, | (Alachua, FL, USA) |
| Na(BARF)₄ | |
| Allyldichloro(1,3-bis(2,6- | Strem (Newburyport, MA, USA) |
| diisopropyl-phenyl)imidazol- | |
| 2-ylidene)palladium (II), | |
| DippPd(allyl)Cl | |
| 1,2-Dichloroethane | Alfa Aesar (Ward Hill, MA, USA) |
| Irgacure 819 photoinitiator | BASF Corp (Florham Park, |
| | NJ, USA) |
| Dicumyl peroxide | Sigma-Aldrich (St. Louis, |
| | MO, USA) |
| Dicyclopentadiene, DiCp | EMD Millipore (Burlington, MA, |
| | USA) |
| Allyl Amine | Alfa Aesar (Ward Hill, MA, USA) |
| Pyridine | Alfa Aesar (Ward Hill, MA, USA) |
| 2-Butanone | EMD Millipore (Burlington, MA, |
| | USA) |

### Synthesis of Norbornene Itaconimide (Mixture A)

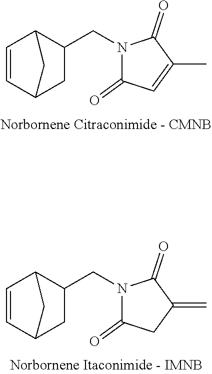 [SMILES: CC1=CC(=O)N(CC2CC3C=CC2C3)C1=O ; C=C1CC(=O)N(CC2CC3C=CC2C3)C1=O]

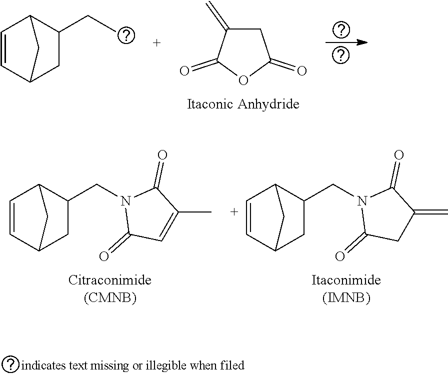

A 250 mL 3-neck round bottom flask equipped with an addition funnel, magnetic stirring, and an N₂ blanket was charged with 16.8 g (0.15 mol) itaconic anhydride and 100 mL toluene. The addition funnel was charged with 18.4 g (0.15 mol) 5-norbornene-2-methylamine, and the liquid was added dropwise to the stirred mixture of toluene and itaconic anhydride with the flask in the oil bath at 90° C. The vial and addition funnel were washed out with an additional 10 mL of toluene which was also added to the reaction mixture. After addition was complete, the addition funnel was removed and replaced with a Dean-Stark trap and a reflux condenser, and with the flask under a nitrogen blanket, the oil bath temperature was gradually raised to 145° C. to start toluene reflux. The apparatus was left to continue refluxing overnight. Upon return, the reaction mixture was a clear reddish-brown solution with a small amount of solid visible on the flask wall. About 1.9 mL of water had collected in the trap after 20 hours of refluxing. The reaction mixture was washed twice with 100 mL portions of 1 N KOH and twice with 100 mL portions of 1 wt. % KCl solution. The toluene solution was dried over anhydrous MgSO₄ and gravity filtered through #4 filter paper to obtain a clear reddish brown solution. Solvent was removed by rotary evaporation then a gentle nitrogen flush to leave a clear red-brown liquid.

The liquid was purified by Kugelrohr vacuum distillation at 140-185° C. and 500 mTorr, yielding a crystal clear, colorless liquid distillate. Analysis of the distillate by ¹H NMR in CDCl₃ solution showed a clean mixture of 75% CMNB and 25% IMNB.

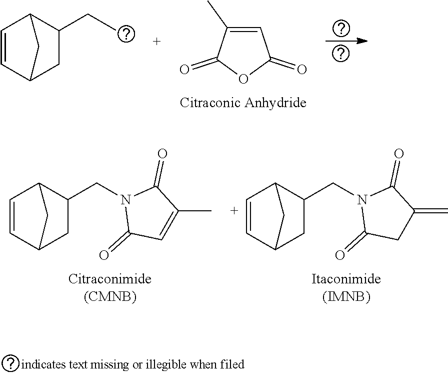

A 250 mL 3-neck round bottom flask equipped with an addition funnel, magnetic stirring and an N₂ blanket was charged with 16.8 g (0.15 mol) citraconic anhydride and 90 mL toluene. The mixture was stirred to dissolve the anhydride. The addition funnel was charged with 18.4 g (0.15 mol) of 5-norbornene-2-methylamine which was then added dropwise to the mixture. The funnel was then rinsed with 20 mL of toluene. Magnetic stirring was maintained as the mixture turned hazy and solidified due to the precipitation of the amic acid adduct. After addition was complete, the flask was fitted with a Dean-Stark trap and condenser and transferred into an oil bath at 90° C. which was then gradually raised to 145° C. to begin refluxing the toluene. After a few hours, the reaction mixture melted out. Reflux was continued for a total of 21 hours. This left a clear dark brown liquid reaction mixture with no trace of precipitate. 2.1 mL had collected in the trap. The reaction mixture was cooled to room temperature, then put in a separatory funnel and washed twice with 100 mL portions of 5 wt. % KHCO₃ solution and once with 100 mL of 2 wt. % KCl solution. The toluene phase was dried over anhydrous MgSO₄ and filtered through #4 filter paper into a 250 mL round bottom flask. Solvent was removed by rotary evaporation, leaving a clear dark brown liquid.

The liquid was purified by Kugelrohr vacuum distillation at 145-185° C. and 1 Torr, yielding a light-yellow liquid distillate. Analysis of the distillate by ¹H NMR in CDCl₃ solution showed a clean mixture of 90% CMNB and 10% IMNB.

### Synthesis of Polymers

In a 20 mL vial, a catalyst solution was prepared by dissolving allyldichloro(1,3-bis(2,6-diisopropyl-phenyl)imidazol-2-ylidene)palladium (II), tricyclohexylphosphine, and sodium tetrakis [3,5-bis(trifluoromethyl)phenyl]borate into 1,2 dichloroethane in the amounts listed in Table 2. This yielded a clear light-yellow solution.

TABLE 2
Composition of catalyst solutions for each example

| | | | | 1,2- |
|---|---|---|---|---|
| | DippPd(allyl)Cl | Na(BARF)4 | PCy₃ | Dichloroethane |
| Example | mg (μmol) | mg (μmol) | mg (μmol) | g |
| 1 | 15.2 (27) | 118 (133) | 14.9 (53) | 16.82 |
| 2 | 15.2 (27) | 118 (133) | 14.9 (53) | 16.82 |
| 3 | 22.9 (40) | 177 (200) | 22.4 (80) | 25.30 |
| 4 | 22.9 (40) | 177 (200) | 22.4 (80) | 25.30 |

In a 500 mL 3-neck round bottom flask equipped with a paddle blade stir rod and an N2 blanket, norbornene (NB), hexylnorbornene (HNB), the mixture of IMNB and CMNB, and an equimolar amount of 1-octene were dissolved in toluene in the amounts listed in Table 3. The contents were then stirred using a model 850 electric mixer by Arrow Engineering (Hillside, NJ, USA) at a setting of 2 using the analog dial.

TABLE 3
Composition of monomer mixtures for each sample

| | | | | | Mixture B | | | |
|---|---|---|---|---|---|---|---|---|
| | | HNB | Mixture A | | | CMNB | | |
| | NB | g | IMNB | CMNB | IMNB | g | 1-Octene | Toluene |
| Example | g (mmol) | (mmol) | g (mmol) | g (mmol) | g (mmol) | (mmol) | g (mmol) | g |
| 1 | | 17.83 | 1.79 | 5.38 | | | 14.93 | 63.31 |
| | | (100) | (8) | (25) | | | (133) | |
| 2 | | 17.83 | | | 0.72 | 6.45 | 14.93 | 63.31 |
| | | (100) | | | (3) | (30) | (133) | |
| 3 | 8.47 | 16.05 | 1.09 | 3.26 | | | 22.44 | 73.56 |
| | (90) | (90) | (5) | (15) | | | (200) | |
| 4 | 8.47 | 16.05 | | | 0.43 | 3.91 | 22.44 | 73.56 |
| | (90) | (90) | | | (2) | (18) | (200) | |

Using a syringe with a 20-gauge needle tip, the catalyst solution was drawn up out of the vial and injected into the round bottom flask with the monomer mixture while it was stirring. The addition of the catalyst solution initiated the reaction of the monomers. The contents were allowed to polymerize at room temperature while mixing for several days.

The reaction was terminated by pipetting roughly 1 mL of pyridine into the polymerized mixture while it was stirring. The pyridine quenches the catalyst, inactivating it. The polymer was isolated out of the reaction mixture by very slowly pouring it into a rapidly stirring large beaker (using the same paddle blade stir rod) containing 2 L of 2-butanone. The 2-butanone acted as a nonsolvent which immediately precipitated the polymer into a fine string because of the rapid stirring. Once all the reaction mixture was added, 2-butanone was added to the round bottom flask to precipitate out any remaining reaction mixture still in the flask. That solid was then transferred to the stirring beaker. With all the contents now in the beaker, the stirring was stopped and the paddle blade stir rod was removed. The polymer had wrapped itself around the stir blade, so a razor blade and scissors were used to help break up the polymer into smaller pieces and aid in the removal. The beaker was then covered with aluminum foil and transferred to a refrigerator to chill overnight. The next day, the solid material was isolated out of the nonsolvent by vacuum filtration using a Buchner funnel. The solid material was then transferred to a large aluminum pan, which was then covered and placed in an oven to dry at 90° C. for 4 hours. Table 4 lists the reaction time and yield for each example.

TABLE 4
Reaction time and % yield for each example

| | Reaction | Yield |
|---|---|---|
| Example | Time hrs | % |
| 1 | 166 | 24.6 |
| 2 | 144 | 34.5 |
| 3 | 96 | 86.6 |
| 4 | 94 | 86.9 |

### Film Formation

In separate 4 oz jars, roughly 2-5 g of each polymer example (Example 1-4) was added to enough toluene to make 10-20% solids solutions. The jars were then sealed and placed on a jar roller and allowed to roll overnight until the polymer was completely dissolved. The solutions were then poured into small glass Petri dishes. The dishes were placed uncovered in a cabinet and the toluene was allowed to evaporate out for about 3 days yielding freestanding films.

For some samples, 2 parts per hundred resin (PHR) of dicumyl peroxide thermal initiator or 1 PHR of Irgacure 819 photoinitiator was added to the jar to dissolve in with the polymer sample.

### Crosslinking the Films

Films that were thermally crosslinked were stacked between sheets of PTFE film and sandwiched between 2 heavy metal plates to keep the film flat. The stack of films was then placed in a N₂ purged oven at room temperature. Using a temperature controller, the temperature of the oven was ramped up to 200° C. at a rate of 5° C./min. Once the temperature had reached 200° C., it was held at that temperature for 2 hours before being cooled down to room temperature again.

Films that were photo-crosslinked were cured using a Fusion UV Light Hammer LHC10 Mark II (Heraeus Noblelight America, Gaithersburg, MD, USA) equipped with a D-bulb. The films were passed through the processor twice at 21 ft/min with the bulb at 100% power which delivered a total energy of 4.1 J/cm² UVA, 1.3 J/cm² UVB, 0.6 J/cm² UVC and 5.0 J/cm² UVV as measured by an EIT PowerPuck II (EIT, Leesburg, VA, USA). Details of the conditions and compositions for the crosslinked films can be found in Table 5.

TABLE 5
Condition and composition of crosslinked films

| | Starting | | Initiator | Curing |
|---|---|---|---|---|
| Example | Polymer | Initiator | loading (PHR) | Condition |
| 5 | Example 1 | Dicumyl peroxide | 2 | Thermal cure |
| 6 | Example 2 | Dicumyl peroxide | 2 | Thermal cure |
| 7 | Example 3 | Dicumyl peroxide | 2 | Thermal cure |
| 8 | Example 4 | Dicumyl peroxide | 2 | Thermal cure |
| 9 | Example 1 | Irgacure 819 | 1 | Photo-cure |
| 10 | Example 2 | Irgacure 819 | 1 | Photo-cure |
| 11 | Example 3 | Irgacure 819 | 1 | Photo-cure |
| 12 | Example 4 | Irgacure 819 | 1 | Photo-cure |

### Mechanical Property Evaluation Using DMA

Dynamic mechanical analysis was performed on a TA Instruments RSA-G2 DMA (New Castle, DE, USA) using a temperature ramp with oscillating strain using the following parameters: Equilibrate at −30° C.; Isothermal for 3 min; Ramp 3° C./min to 300° C.; Strain=0.1%; Frequency=1 Hz. Data are reported in FIGS. 13-16 and summarized in Table 6.

TABLE 6
Comparison of storage modulus, E′, at high and low temps indicating
performance of crosslinking (IMNB and CMNB content (mole fraction)
calculated based on starting monomer composition)

| | | | | | | E′ @ | |
|---|---|---|---|---|---|---|---|
| | | IMNB | CMNB | E′ @ 30 | E′ @ 280 | 30 °C/ | |
| | | Content | Content | °C | °C | E′ @ | Tg |
| Example | | mol % | mol % | Mpa | Mpa | 280 °C | °C |
| Uncured | 1 | 6.2 | 18.8 | 791 | 9.74 | 81.2 | 250 |
| | 2 | 2.5 | 22.5 | 787 | 5.93 | 133 | 249 |
| | 3 | 2.5 | 7.5 | 1199 | 3.42 | 351 | 231 |
| | 4 | 1.0 | 9.0 | 1205 | 3.18 | 379 | 233 |
| Thermal | 5 | 6.2 | 18.8 | 761 | 72.5 | 10.5 | 239 |
| cure | 6 | 2.5 | 22.5 | 702 | 71.4 | 9.83 | 254 |
| | 7 | 2.5 | 7.5 | 1213 | 7.97 | 152 | 237 |
| | 8 | 1.0 | 9.0 | 1264 | 8.42 | 150 | 237 |
| Photo-cure | 9 | 6.2 | 18.8 | 787 | 32.4 | 24.3 | 264 |
| | 10 | 2.5 | 22.5 | 766 | 27.1 | 28.3 | 262 |
| | 11 | 2.5 | 7.5 | 1403 | 5.47 | 256 | 240 |
| | 12 | 1.0 | 9.0 | 1205 | 3.43 | 351 | 239 |

The DMA data reveals differences in the crosslinking of the films effected by the different IMNB/CMNB ratios. The amount of crosslinking can be inferred by examining the ratio of storage modulus, E′, at low (30° C.) and high (280° C.) temperatures, quantified as E′_{30° C.}/E′_{280° C.} Example 2/Example 1 and Example 4/Example 3 are two good comparisons to show the impact of increased IMNB; these pairs have similar composition of NB, HNB and amide monomer but differ in IMNB/CMNB ratio. In both instances, the increased amount of IMNB leads to a lower E′_{30° C.}/E′_{280° C.}, 133 to 81.2 for Example 2/Example 1 and 379 to 351 for Example 4/Example 3. This observation remained true for the photo-cured examples but not for the thermally cured examples. However, the E′_{30° C.}/E′_{280° C.} ratios of the thermally cured samples were quite similar.

Another useful comparison is between samples with the same IMNB/CMNB ratio but increased amount of these crosslinkable monomers, as in Example 3/Example 1 and Example 4/Example 2. Both cases show a higher degree of crosslinking in the samples with more IMNB/CMNB monomer, with E′_{30° C.}/E′_{280° C.} of 351 to 81.2 for Example 3/Example 1 and 379 to 133 for Example 4/Example 2. This observation was true for all examples. These data clearly show the benefit of higher levels of the IMNB monomer for improving crosslinking in these thermoset systems.

### Dielectric Property Evaluation

### Split Post Dielectric Resonator Measurements

All split-post dielectric resonator measurements were performed in accordance with the standard IEC 61189-2-721, at 10.1, 15.22 and 24.7 GHz, and 25° C. Each material was inserted between two fixed dielectric resonators. The resonance frequency and quality factor of the posts are influenced by the presence of the specimen, and this enables the direct computation of complex permittivity (dielectric constant (ε′) and dielectric loss (ε″)). The geometry of the split dielectric resonator fixture used in our measurements was designed by the Company QWED in Warsaw Poland. These resonators operate with the TE01d mode which has only an azimuthal electric field component so that the electric field remains continuous on the dielectric interfaces. The split post dielectric resonator measures the permittivity component in the plane of the specimen. Loop coupling (critically coupled) was used in each of these dielectric resonator measurements. This Split Post Resonator measurement system was combined with Keysight VNA (Vector Network Analyzer Model PNA N5222B along with millimeter-wave test set model N5292A, 900 Hz-110 GHz). Computations were performed with the commercial analysis Split Post Resonator Software of QWED to provide a powerful measurement tool for the determination of complex electric permittivity of each specimen at the specific frequency. Tan delta (tan δ) is also known in the art as the dissipation factor.

TABLE 7
Dielectric Properties

| | Thickness | Frequency | | | |
|---|---|---|---|---|---|
| Example | (mm) | (GHz) | ε′ | ε″ | tan(δ) |
| 3 | 0.3068 | 10.1 | 2.31453 | 0.00252 | 0.001089 |
| | 0.3072 | 15.22 | 2.34307 | 0.00273 | 0.001165 |
| | 0.3072 | 24.7 | 2.34031 | 0.00245 | 0.001047 |
| 4 | 0.3392 | 10.1 | 2.30609 | 0.00237 | 0.001028 |
| | 0.3392 | 15.22 | 2.33555 | 0.00254 | 0.001088 |
| | 0.3392 | 24.7 | 2.28069 | 0.00226 | 0.000991 |

Foreseeable modifications and alterations of this invention will be apparent to those skilled in the art without departing from the scope and spirit of this invention. This invention should not be restricted to the embodiments that are set forth in this application for illustrative purposes.

1. A composition comprising a mixture of isomers comprising:
at least 10% by weight of isomer A, which has the following formula: 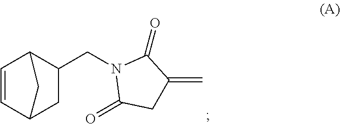 [SMILES: C=C1CC(=O)N(CC2CC3C=CC2C3)C1=O]
and
up to 90% by weight of isomer B, which has the following formula:

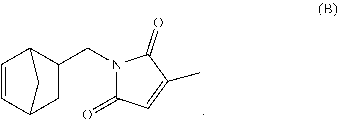 [SMILES: CC1=CC(=O)N(CC2CC3C=CC2C3)C1=O]

2. The composition of claim 1, wherein the isomer A is present in an amount of 10% by weight (wt. %) to 75 wt. % of the mixture of isomers and the isomer B is present in an amount of 25 wt. % to 90 wt. % of the mixture of isomers.

3. The composition of claim 1, wherein the isomer A and the isomer B together comprise 95 wt. % to 100 wt. % of the total composition.

4. A copolymer comprising the following divalent monomer units: 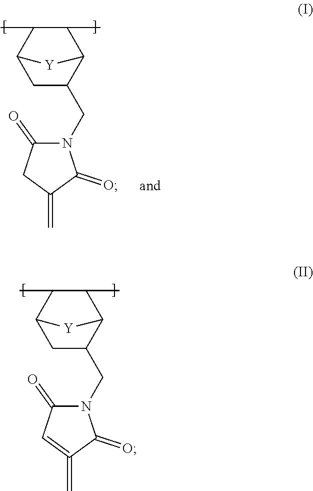
wherein each Y is independently selected from —CH₂—, —CH₂CH₂—, and O.

5. A copolymer comprising the following divalent monomer units: 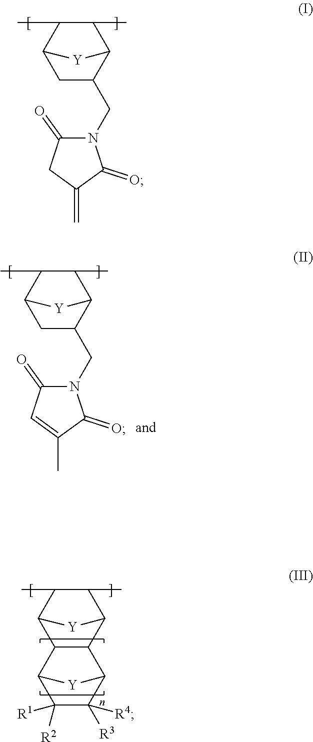
wherein each of R¹, R², R³, and R⁴ is independently selected from H, a linear or branched C1 to C20 hydrocarbyl, a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

6. The copolymer of claim 5, wherein in the divalent unit of formula (III), at least one of R¹, R², R³, and R⁴ is a C1 to C7 linear alkyl, vinyl, or a C1 to C20 carbosilane.

7. The copolymer of claim 5, wherein in the divalent unit of formula (III), each of R¹, R², R³, and R⁴ is H; n is 0; and Y is —CH₂—.

8. The copolymer of claim 5, wherein in the divalent unit of formula (III), one of R¹, R², R³, and R⁴ is a C6 alkyl group; each of the rest of R¹, R², R³, and R⁴ is H; n is 0; and Y is —CH₂—.

9. The copolymer of claim 5, comprising at least two divalent units of formula (III) that are different from each other.

10. The copolymer of claim 9, wherein in a first of the divalent units of formula (III), each of R¹, R², R³, and R⁴ is H; n is 0; and Y is —CH₂—; and in a second of the divalent units of formula (III), one of R¹, R², R³, and R⁴ is a C6 alkyl group and each of the rest of R¹, R², R³, and R⁴ is H; n is 0; and Y is —CH₂—.

11. A copolymer that is a reaction product of a polymerizable composition comprising: 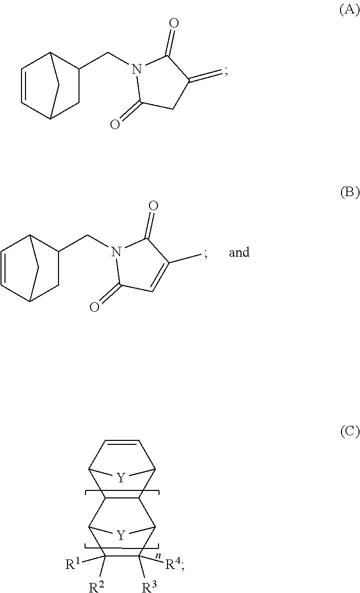
wherein each of R¹, R², R³, and R⁴ is independently selected from H, a linear or branched C1 to C20 hydrocarbyl; a linear or branched C1 to C20 heterohydrocarbyl, a C1 to C20 carbosilane, and a C5 to C20 heterocyclic ring, or R¹ and R² taken together or R³ and R⁴ taken together form a C1 to C20 hydrocarbylidene group; n is an integer of 0 to 5; and wherein each Y is independently selected from —CH₂—, —CH₂CH₂—, and O.

12. The copolymer of claim 11, wherein the polymerizable composition further comprises a chain transfer agent.

13. The copolymer of claim 11, comprising at least two monomers of formula (C) that are different from each other, wherein in a first of the monomers of formula (C), each of R¹, R², R³, and R⁴ is H; n is 0; and Y is —CH₂— and in a second of the monomers of formula (C), one of R¹, R², R³, and R⁴ is a C6 alkyl group; each of the rest of R¹, R², R³, and R⁴ is H; n is 0; and Y is —CH₂—.

14. The copolymer of claim 4, wherein the copolymer is a form of a film.

15. A composition comprising the copolymer of claim 4 and a solvent.

16. The composition of claim 15, further comprising at least one of a sensitizer, an initiator, a filler, an antioxidant, an inhibitor, a stabilizer, a colorant, or an adhesion control agent.

17. The composition of claim 16, wherein the initiator comprises at least one of a thermal initiator or a photoinitiator.

18. A film comprising a crosslinked reaction product of the copolymer of claim 4.
